(19) United States
(12) Patent Application Publication
(10) Pub. No.: US 2024/0082200 A1
(43) Pub. Date: Mar. 14, 2024

(54) 4-THIOCARBAMOYLPHENYL 2-(4-OXOPHENYL)-PHENYL PROPANOATE FOR THE TREATMENT OF PAIN ASSOCIATED WITH UROLOGIC CHRONIC PELVIC PAIN SYNDROME

(71) Applicant: Antibe Therapeutics Inc., Toronto (CA)

(72) Inventors: John L. Wallace, Toronto (CA); Marcelo N. Muscará, São Paulo (BR); Soraia Kátia Pereira Costa, São Paulo (BR); David Vaughan, Toronto (CA)

(21) Appl. No.: 18/237,675

(22) Filed: Aug. 24, 2023

Related U.S. Application Data
(60) Provisional application No. 63/401,327, filed on Aug. 26, 2022.

(51) Int. Cl. A61K 31/216; A61P 29/00

(52) U.S. Cl. CPC A61K 31/216; A61P 29/00

(57) ABSTRACT

The present application relates to methods of treating urologic chronic pelvic pain syndrome (UCPPS) or pain associated with UCPPS using a compound of Formula I, or a pharmaceutically acceptable salt, prodrug and/or solvate thereof. UCPPS encompasses interstitial cystitis/bladder pain syndrome (IC/BPS) and chronic prostatitis/chronic pelvic pain syndrome (CP/CPPS).

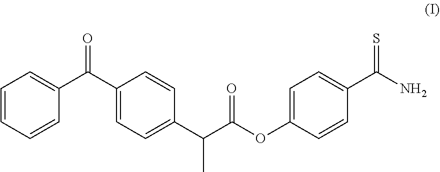 [SMILES: CC(C(=O)Oc1ccc(C(N)=S)cc1)c1ccc(C(=O)c2ccccc2)cc1]

### RELATED APPLICATIONS

The present application claims the benefit of priority of co-pending U.S. provisional patent application No. 63/401,327, filed on Aug. 26, 2022, the contents of which are incorporated herein by reference in its entirety.

### FIELD

The present application relates to methods of treating or preventing pain associated with urologic chronic pelvic pain syndrome (UCPPS) using 4-thiocarbamoylphenyl 2-(4-oxophenyl)-phenyl propanoate, or a pharmaceutically acceptable salt, prodrug and/or solvate thereof. UCPPS includes interstitial cystitis/bladder pain syndrome (IC/BPS) and chronic prostatitis/chronic pelvic pain syndrome (CP/CPPS).

### BACKGROUND

NSAIDs along with opioids, anticonvulsants and antimigraine drugs such as 5-HT receptor agonists and ergot derivatives are routinely used for the treatment of acute and chronic pain. Implementation of nonsteroidal anti-inflammatory drugs (NSAIDs), such as ketoprofen, is known in clinical pharmacology to be associated with a variety of gastrointestinal (GI) side effects, including mild or severe dyspeptic symptoms, development of gastric or duodenal ulcers, hemorrhage, perforations, and other events (Rusell R. I., 2001). Among others, NSAIDs are used in the treatment of chronic inflammation in patients with rheumatoid arthritis, however, very often inducing gastritis (Bindu S et al., 2020). One of the most important mechanisms underlying NSAID-induced GI damage involves but is not limited to the inhibition of cyclooxygenase (COX)-1 or COX-2 producing gastroprotective prostaglandins (PGs), such as PGE₂ (Bindu S et al., 2020, Glowacka et al., 2020). These drugs are also generally short-acting thus requiring repeated dosing. Therefore, there is a need for more efficacious analgesics for the treatment of pain. For example, urologic chronic pelvic pain syndrome (UCPPS) is a challenging painful condition which encompasses interstitial cystitis/bladder pain syndrome (IC/BPS) and chronic prostatitis/chronic pelvic pain syndrome (CP/CPPS). People afflicted with UCPPS often experience pain in the pelvic region that can cause significant discomfort and disability.

Hydrogen sulphide (H₂S) is produced by any cell type, including in the GI tract where it has been found to contribute to gastro-intestinal mucosal defense and the healing of ulcers. It is an endogenous antioxidative gaseous mediator shown to be involved in digestive system physiology, to accelerate gastric ulcer healing and to prevent GI tract against the damage induced by stress, ischemia/reperfusion, and drugs, including NSAIDs (Lasheen N N et al., 2019, Sun H., et al 2020; Wallace J. L., et al. 2017).

U.S. Pat. No. 8,541,398 discloses NSAID derivatives covalently linked to H₂S-releasing moieties and found that the anti-inflammatory effects of a variety of NSAIDs are significantly enhanced when covalently linked to a H₂S-releasing moiety, or when NSAID salts are formed with an H₂S-releasing moiety. Further, U.S. Pat. No. 8,541,398 discloses that these NSAID derivatives covalently linked to H₂S-releasing moieties deliver potent anti-inflammatory properties without causing gastro-intestinal toxicity. Of the many NSAID derivatives covalently linked to H₂S-releasing moieties, U.S. Pat. No. 8,541,398 discloses a hydrogen sulfide-releasing derivative of ketoprofen chemically known as 4-thiocarbamoylphenyl 2-(4-oxophenyl)-phenyl propanoate, and sometimes known as ATB-352 (Antibe Therapeutics Inc., Toronto, Canada). ATB-352 has been shown to be more potent and effective in periodontitis, an inflammatory condition, than its parent drug (Gugliadola et al., 2018), and to have increased GI safety compared to ketoprofen (Glowacka et al., 2022). ATB-352 has also been shown to have increased efficacy over over ketoprofen in surgical incision pain model in mice (Costa et al., 2020).

### SUMMARY

The Applicant has shown that the compound of Formula I, an NSAID derivative covalently linked to H₂S-releasing moiety wherein the NSAID is ketoprofen and the H₂S-releasing moiety is 4-hydroxythiobenzamide (TBZ), and sometimes known as ATB-352, can alleviate bladder inflammation and pain associated with urologic chronic pelvic pain syndrome.

Accordingly, the present application includes a method of treating pain associated with urologic chronic pelvic pain syndrome (UCPPS) in a subject in need thereof comprising administering, to the subject, an effective amount of a compound of Formula I,

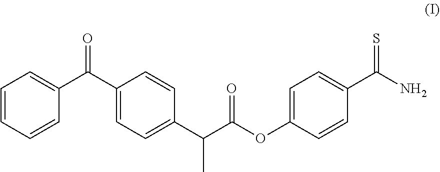 [SMILES: CC(C(=O)Oc1ccc(C(N)=S)cc1)c1ccc(C(=O)c2ccccc2)cc1]

or a pharmaceutically acceptable salt, prodrug and/or solvate thereof.

In some embodiments, the pain associated with UCPPS is selected from one or more of visceral pain, neuropathic pain, neuralgia, allodynia, causalgia, hyperalgesia, hyperesthesia, hyperpathia and neuropathy secondary to surgical procedure.

In some embodiments, the pain associated with UCPPS is acute pain associated with UCPPS.

The present application also includes a method of treating urologic chronic pelvic pain syndrome (UCPPS) in a subject in need thereof comprising administering, to the subject, an effective amount of a compound of Formula I,

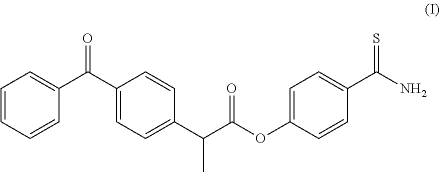 [SMILES: CC(C(=O)Oc1ccc(C(N)=S)cc1)c1ccc(C(=O)c2ccccc2)cc1]

or a pharmaceutically acceptable salt, prodrug and/or solvate thereof.

The present application further includes a method of treating one or more symptoms associated with urologic chronic pelvic pain syndrome (UCPPS) in a subject in need thereof comprising administering, to the subject, an effective amount of a compound of Formula I,

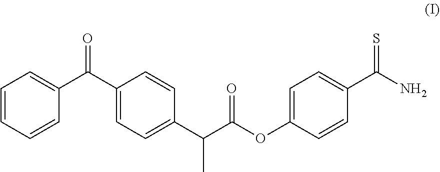 [SMILES: CC(C(=O)Oc1ccc(C(N)=S)cc1)c1ccc(C(=O)c2ccccc2)cc1]

or a pharmaceutically acceptable salt, prodrug and/or solvate thereof.

The present application also includes a method of treating bladder inflammation in a subject in need thereof comprising administering, to the subject, an effective amount of a compound of Formula I,

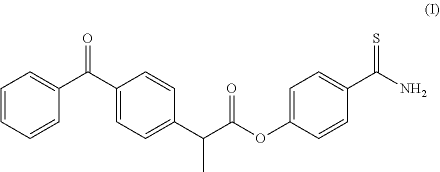 [SMILES: CC(C(=O)Oc1ccc(C(N)=S)cc1)c1ccc(C(=O)c2ccccc2)cc1]

or a pharmaceutically acceptable salt, prodrug and/or solvate thereof.

The present application also includes a method of increasing urinary bladder contraction and relaxation in a subject in need thereof comprising administering, to the subject, an effective amount of a compound of Formula I,

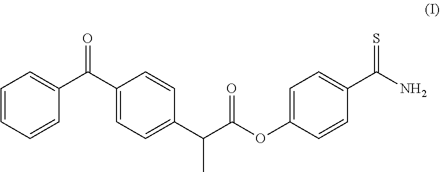 [SMILES: CC(C(=O)Oc1ccc(C(N)=S)cc1)c1ccc(C(=O)c2ccccc2)cc1]

or a pharmaceutically acceptable salt, prodrug and/or solvate thereof.

The present application further includes a method of treating acute pain in a subject in need thereof comprising administering, to the subject, an effective amount of a compound of Formula I,

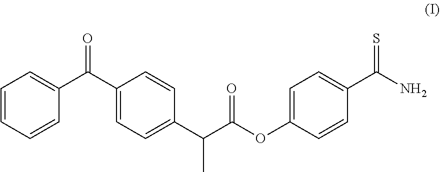 [SMILES: CC(C(=O)Oc1ccc(C(N)=S)cc1)c1ccc(C(=O)c2ccccc2)cc1]

or a pharmaceutically acceptable salt, prodrug and/or solvate thereof.

In some embodiments, the UCPPS includes interstitial cystitis/bladder pain syndrome (IC/BPS) and chronic prostatitis/chronic pelvic pain syndrome (CP/CPPS).

In some embodiments, the compound of Formula I or a pharmaceutically acceptable salt, prodrug and/or solvate thereof is formulated into a pharmaceutical composition comprising a compound of Formula I or a pharmaceutically acceptable salt, prodrug and/or solvate thereof and one or more pharmaceutically carriers.

Other features and advantages of the present application will become apparent from the following detailed description. It should be understood, however, that the detailed description and the specific examples, while indicating embodiments of the application, are given by way of illustration only. The scope of the claims should not be limited by these embodiments; rather, they should be given the broadest interpretation consistent with the description as a whole.

### DRAWINGS

The embodiments of the application will now be described in greater detail with reference to the attached drawings in which:

FIG. 1 is a graph showing comparative analgesic effects of compound of Formula I (ATB-352) and ketoprofen in a mouse model of visceral pair (cyclophosphamide (CYP)-induced interstitial cystitis). Mice were treated orally with compound of Formula I (4.6, 15, 46 or 77 mg kg′), equimolar doses of ketoprofen (3, 10, or 50 mg kg′) or vehicle (CMC 05%) 21 h after induction of cystitis. Data are expressed as area-under-the-curve for the interval of 21 to 24 hours after the treatment with the test compounds. Data are expressed as mean±SEM (n=5−7). *P<0.05 vs. vehicle. #P<0.05 vs. CYP group, and expP<0.001 vs. equimolar doses of ketoprofen. One-way ANOVA followed by Tukey's test.

FIG. 2 shows the induction of gastric mucosal hemorrhagic injury by ketoprofen, but not by the compound of Formula I (ATB-352). The area (mm²) of hemorrhagic gastric lesions was evaluated 3 hours after oral administration of vehicle (0.5% carboxymethylcellulose, 10 ml kg⁻¹), compound of formula I (77 mg kg⁻¹) or ketoprofen (50 mg kg⁻¹) using computerized planimetry software (ImageJ®). ⁽¹⁾P<0.05 comparing to the compound of Formula I group (one-way ANOVA followed by Tukey's test).

FIG. 3 shows the comparative effects of compound of Formula I (ATB-352) and ketoprofen on the reversal of CYP induced reduced bladder contractility in responses to KCI and CCh. (A) shows the contractile response to KCI (80 mM) and (B) carbachol maximal contraction response in bladder from vehicle, CYP, compound of Formula I (4.6, 15, 46 and 77 mg kg⁻¹) and ketoprofen (3, 10, 30 or 50 mg kg⁻¹) groups. Data are expressed as mean±SEM (n=4−7). *P<0.05 vs. vehicle. #P<0.05 vs. CYP. ##P<0.001 vs. vehicle (A and B: one-way ANOVA followed by Tukey's test).

### DETAILED DESCRIPTION

### I. Definitions

Unless otherwise indicated, the definitions and embodiments described in this section and in other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed terms that specify the presence of the stated features, elements, components, groups, integers, and/or steps, but exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least±5% of the modified term if this deviation would not negate the meaning of the word it modifies.

As used in this application, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “a compound of Formula I” or “ATB-352” as used herein refers to a compound having the chemical name: 4-thiocarbamoylphenyl 2-(4-oxophenyl)-phenyl propanoate, and having the chemical Formula

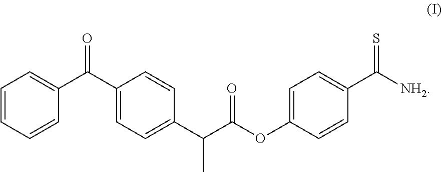 [SMILES: CC(C(=O)Oc1ccc(C(N)=S)cc1)c1ccc(C(=O)c2ccccc2)cc1]

The term “ketoprofen” as used herein refers to a compound having the chemical name: m-benzoylhydratropic acid or 2-(4-benzoylphenyl)propanoic acid, and having the chemical Formula:

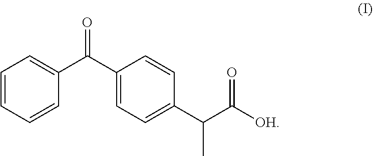 [SMILES: CC(C(=O)O)c1ccc(C(=O)c2ccccc2)cc1]

The term “pharmaceutically acceptable salt” means either an acid addition salt or a base addition salt which is suitable for, or compatible with the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “solvate” as used herein means a compound, or a salt of a compound, wherein molecules of a suitable solvent are incorporated in the crystal lattice. A suitable solvent is physiologically tolerable at the dosage administered.

The term “prodrug” as used herein means a compound, or salt of a compound, that, after administration, is converted into an active drug.

The term “administered” as used herein means administration of a therapeutically effective amount of a compound or a pharmaceutically acceptable salt, prodrug and/or solvate thereof or compositions comprising a compound or a pharmaceutically acceptable salt, prodrug and/or solvate thereof to a cell either in cell culture or in a subject.

As used herein, the term “effective amount”, “therapeutically effective amount” means an amount of a compound of Formula I or a pharmaceutically acceptable salt, prodrug and/or solvate thereof, or compositions comprising a compound of Formula I or a pharmaceutically acceptable salt, prodrug and/or solvate thereof, that is effective, at dosages and for periods of time necessary to achieve the desired result, including therapeutic and prophylactic results.

The term “subject” as used herein includes all members of the animal kingdom, including mammals, and suitably refers to humans. Thus the methods and uses of the present application are applicable to both human therapy and veterinary applications.

The term “pharmaceutical composition” as used herein refers to a composition of matter for pharmaceutical use.

The term “pharmaceutically acceptable” means compatible with the treatment of subjects.

The term “parenteral” as used herein means taken into the body or administered in a manner other than through the gastrointestinal tract.

The terms “to treat”, “treating” and “treatment” as used herein and as is well understood in the art, means an approach for obtaining beneficial or desired results, including clinical results. Examples of beneficial or desired clinical results include, but are not limited to diminishment of extent of disease, stabilized (i.e., not worsening) state of disease, preventing spread of disease, delay or slowing of disease progression, amelioration or palliation of the disease state, and remission (whether partial or total), whether detectable or undetectable, diminishment of the reoccurrence of disease. “To treat”, “treating” and “treatment” can also mean prolonging survival as compared to expected survival if not receiving treatment. “To treat”, “treating” and “treatment” as used herein also include prophylactic treatment.

“Palliating” a disease, disorder or condition means that the extent and/or undesirable clinical manifestations of a disease, disorder or condition are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition or manifesting a symptom associated with a disease, disorder or condition.

The term “treating pain associated with urologic chronic pelvic pain syndrome” as used herein means any detectable reduction, for example, in the sensation or duration of pain associated with urologic chronic pelvic pain syndrome after administration or use of a compound compared to otherwise the same conditions except in the absence of administration or use of the compound.

The term “treating pain associated with interstitial Cystitis/Bladder Pain Syndrome (IC/BPS)” as used herein means any detectable reduction, for example, in the sensation or duration of pain associated with interstitial Cystitis/Bladder Pain Syndrome (IC/BPS) after administration or use of a compound compared to otherwise the same conditions except in the absence of administration or use of the compound.

The term “treating pain associated with chronic prostatitis/chronic pelvic pain syndrome (CP/CPPS)” as used herein means any detectable reduction, for example, in the sensation or duration of pain associated with CP/CPPS after administration or use of a compound compared to otherwise the same conditions except in the absence of administration or use of the compound.

The term “urologic chronic pelvic pain syndrome” or “UCPPS” as used herein refers to a chronic pain condition in the pelvis, prostate, bladder, and/or genitalia often accompanied by urinary frequency and urgency. UCPPS encompasses interstitial cystitis/bladder pain syndrome and chronic prostatitis/chronic pelvic pain syndrome.

The term “interstitial Cystitis/Bladder Pain Syndrome” or “IC/BPS” as used herein refers to persistent or recurrent chronic pelvic pain, pressure or discomfort perceived to be related to the bladder accompanied by at least one other urinary symptom, such as urgency or frequency.

The term “chronic prostatitis/chronic pelvic pain syndrome” or “CP/CPPS” as used herein refers to long-term pelvic pain and lower urinary tract symptoms (LUTS) without evidence of a bacterial infection.

The term “lower urinary tract symptoms” or “LUTS” as used herein refers to a group of clinical symptoms involving the bladder, urinary sphincter, urethra and, in men, the prostate.

The term “pain associated with urologic chronic pelvic pain syndrome” refers to pain that is directly or indirectly caused by or associated with urologic chronic pelvic pain syndrome.

The term “pain associated with interstitial cystitis/bladder pain syndrome” refers to pain that is directly or indirectly caused by interstitial cystitis/bladder pain syndrome.

The term “pain associated with chronic prostatitis/chronic pelvic pain syndrome” refers to pain that is directly or indirectly caused by chronic prostatitis/chronic pelvic pain syndrome.

The term “acute pain” as used herein means pain that has a sudden onset and is in response to stimuli associated for example with surgery, trauma and/or acute illness that lasts or is anticipated to last a short time (e.g., weeks, days, hours). The term “acute pain” includes, but is not limited to, mild to moderate, moderate, moderate to moderately severe or moderate to severe acute pain.

The term “chronic pain” as used herein means pain associated with a chronic disorder such as UCPPS.

The term “visceral pain” as used herein refers to any pain felt by a subject secondary to a disease, disorder, or condition of an internal organ.

The term “allodynia” as used herein refers to pain due to a stimulus which does not normally provoke pain.

The term “causalgia” as used herein refers to a syndrome of sustained burning pain, allodynia and hyperpathia after a traumatic nerve lesion, often combined with vasomotor and sudomotor dysfunction and later trophic changes.

The term “hyperalgesia” as used herein refers to increased response to a stimulus which is normally painful.

The term “hyperpathia” as used herein refers to a painful syndrome characterized by an abnormally painful reaction to a stimulus, especially a repetitive stimulus, as well as an increased pain threshold.

The term “increasing urinary bladder contraction and relaxation” as used herein refer to any detectable increase in bladder contractile function after administration or use of a compound compared to otherwise the same conditions except in the absence of administration or use of the compound.

The term “treating acute pain” as used herein means any detectable reduction, for example, in the sensation or duration of acute pain after administration or use of a compound, compared to otherwise the same conditions except in the absence of administration or use of the compound.

The term “transient exacerbation” or “flare-up” as used herein in reference to a chronic pain condition refers to an increase in pain intensity.

When used, for example, with respect to the methods of treatment, uses, compositions and kits of the application, a subject, for example a subject “in need thereof” is a subject who has been diagnosed with, is suspected of having, and/or was previously treated for, for example, urologic chronic pelvic pain syndrome” or pain associated with urologic chronic pelvic pain syndrome or bladder inflammation.

### II. Methods and uses of the application

The Applicant has shown that the compound of Formula I, an NSAID derivative covalently linked to H₂S-releasing moiety wherein the NSAID is ketoprofen and the H₂S-releasing moiety is 4-hydroxythiobenzamide (TBZ), and sometimes known as ATB-352, can alleviate bladder inflammation and pain associated with urologic chronic pelvic pain syndrome. For example, the Applicant has studied the acute effects of the compound of Formula I on inflammatory, visceral bladder pain and bladder function and has shown that the compound of Formula I can alleviate visceral bladder and/or pelvic pain and bladder inflammation in an established mouse model of interstitial cystitis/bladder pain syndrome (IC/BPS). The compound of Formula I was also shown to improve bladder contractive function in the IC/BPS model. Surprisingly, the efficacy of the compound of Formula I for alleviating visceral bladder and/or pelvic pain and bladder inflammation, and for improving bladder contractive function in the IC/BPS model was found to be greater than the parent ketoprofen compound.

Accordingly, the present application includes a method of treating pain associated with urologic chronic pelvic pain syndrome (UCPPS) in a subject in need thereof comprising administering, to the subject, an effective amount of a compound of Formula I,

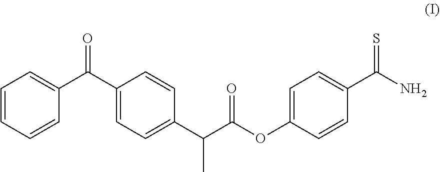 [SMILES: CC(C(=O)Oc1ccc(C(N)=S)cc1)c1ccc(C(=O)c2ccccc2)cc1]

or a pharmaceutically acceptable salt, prodrug and/or solvate thereof.

The present application also includes a use of a compound of Formula I, or a pharmaceutically acceptable salt, prodrug and/or solvate thereof, for treating pain associated with UCPPS in a subject; a use of a compound of Formula I, or a pharmaceutically acceptable salt, prodrug and/or solvate thereof, for preparation of a medicament for treating pain associated with UCPPS in a subject; and compound of Formula I, or a pharmaceutically acceptable salt, prodrug and/or solvate thereof, for use to treat pain associated with UCPPS in a subject.

A person skilled in the art would appreciate that a syndrome characterizes or suggests one more diseases, disorders or conditions. Accordingly, a person skilled in the art would appreciate that UCPPS is a syndrome which includes interstitial cystitis/bladder pain syndrome (IC/BPS) and chronic prostatitis/chronic pelvic pain syndrome (CP/CPPS).

Accordingly, the present application includes a method of treating pain associated with interstitial cystitis/bladder pain syndrome (IC/BPS) in a subject in need thereof comprising administering, to the subject, an effective amount of a compound of Formula I,

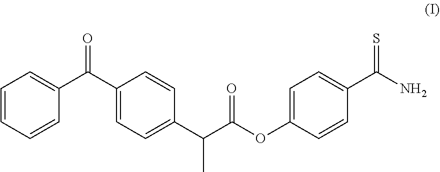 [SMILES: CC(C(=O)Oc1ccc(C(N)=S)cc1)c1ccc(C(=O)c2ccccc2)cc1]

or a pharmaceutically acceptable salt, prodrug and/or solvate thereof.

The present application also includes a use of a compound of Formula I, or a pharmaceutically acceptable salt, prodrug and/or solvate thereof, for treating pain associated with IC/BPS in a subject; a use of a compound of Formula I, or a pharmaceutically acceptable salt, prodrug and/or solvate thereof, for preparation of a medicament for treating pain associated with IC/BPS in a subject; and compound of Formula I, or a pharmaceutically acceptable salt, prodrug and/or solvate thereof, for use to treat pain associated with IC/BPS in a subject.

In some embodiments, the pain is associated with interstitial cystitis. Accordingly, in some embodiments, the compound of Formula I or a pharmaceutically acceptable salt, prodrug and/or solvate thereof is used or administered for treating pain associated with interstitial cystitis.

In some embodiments, the pain is associated with bladder pain syndrome. Accordingly, in some embodiments, the compound of Formula I or a pharmaceutically acceptable salt, prodrug and/or solvate thereof is used or administered for treating pain associated with bladder pain syndrome.

The present application also includes a method of treating pain associated with chronic prostatitis/chronic pelvic pain syndrome (CP/CPPS) in a subject in need thereof comprising administering, to the subject, an effective amount of a compound of Formula I,

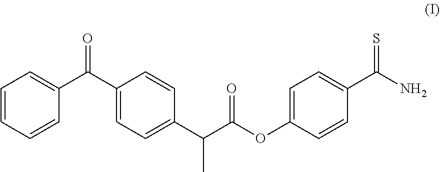 [SMILES: CC(C(=O)Oc1ccc(C(N)=S)cc1)c1ccc(C(=O)c2ccccc2)cc1]

or a pharmaceutically acceptable salt, prodrug and/or solvate thereof.

The present application also includes a use of a compound of Formula I, or a pharmaceutically acceptable salt, prodrug and/or solvate thereof, for treating pain associated with CP/CPPS in a subject; a use of a compound of Formula I, or a pharmaceutically acceptable salt, prodrug and/or solvate thereof, for preparation of a medicament for treating pain associated with CP/CPPS in a subject; and compound of Formula I, or a pharmaceutically acceptable salt, prodrug and/or solvate thereof, for use to treat pain associated with CP/CPPS in a subject.

In some embodiments, the pain associated with urologic chronic pelvic pain syndrome (UCPPS) is selected from one or more of visceral pain, neuropathic pain, neuralgia, allodynia, causalgia, hyperalgesia, hyperesthesia, hyperpathia and neuropathy secondary to surgical procedure. In some embodiments, the UCPPS includes IC/BPS and/or CP/CPPS. Therefore, in some embodiments, the pain associated with IC/BPS is selected from one or more of visceral pain, neuropathic pain, neuralgia, allodynia, causalgia, hyperalgesia, hyperesthesia, hyperpathia and neuropathy secondary to surgical procedure. In some embodiments, the pain associated with CP/CPPS is selected from one or more of visceral pain, neuropathic pain, neuralgia, allodynia, causalgia, hyperalgesia, hyperesthesia, hyperpathia and neuropathy secondary to surgical procedure.

In some embodiments, the pain associated with UCPPS is allodynia associated UCPPS. In some embodiments, the pain associated with IC/BPS is allodynia associated with IC/BPS. In some embodiments, the pain associated with interstitial cystitis is allodynia associated with interstitial cystitis. In some embodiments, the pain associated with bladder pain syndrome is allodynia associated with bladder pain syndrome. In some embodiments, the pain associated with CP/CPPS is allodynia associated with CP/CPPS. In some embodiments, the allodynia associated with UCPPS is abdominal or pelvic allodynia associated with UCPPS. In some embodiments, the allodynia associated with IC/BPS is abdominal or pelvic allodynia associated with IC/BPS. In some embodiments, the allodynia is abdominal or pelvic allodynia associated with IC/BPS. In some embodiments, the allodynia associated with CP/CPPS is abdominal or pelvic allodynia associated with CP/CPPS.

In some embodiments, the pain associated with UCPPS is hyperalgesia associated with UCPPS. In some embodiments, the pain associated with IC/BPS is hyperalgesia associated with IC/BPS. In some embodiments, the pain associated with interstitial cystitis is hyperalgesia associated with interstitial cystitis. In some embodiments, the pain associated hyperalgesia associated with CP/CPPS is hyperalgesia associated with bladder pain syndrome. In some embodiments, the pain associated with CP/CPPS is hyperalgesia associated with CP/CPPS.

In some embodiments, the pain associated with UCPPS is selected from one or more of lower abdominal (pelvic) pain, bladder pain, suprapubic pain, vaginal pain, pain in the penis, testicles, scrotum or perineum, urethral pain, dyspareneuria or pain, pressure or discomfort that may increase as the bladder fills. In some embodiments, the UCPPS includes IC/BPS and/or CP/CPPS.

In some embodiments, the pain associated with UCPPS is visceral pain associated with UCPPS. In some embodiments, the pain associated with interstitial cystitis/bladder pain syndrome (IC/BPS) is visceral pain associated with interstitial cystitis/bladder pain syndrome (IC/BPS). In some embodiments, the pain associated with interstitial cystitis is visceral pain associated with interstitial cystitis. In some embodiments, the pain associated with bladder pain syndrome is visceral pain associated with bladder pain syndrome. In some embodiments, the pain associated with CP/CPPS is visceral pain associated with CP/CPPS. In some embodiments, the visceral pain is visceral abdominal pain.

In some embodiments, the pain associated with urologic chronic pelvic pain syndrome (UCPPS) is acute and/or chronic pain associated with urologic chronic pelvic pain syndrome (UCPPS). In some embodiments, the pain associated with interstitial cystitis/bladder pain syndrome (IC/BPS) is acute and/or chronic pain associated with interstitial cystitis/bladder pain syndrome (IC/BPS). In some embodiments, the pain associated with interstitial cystitis is acute and/or chronic pain associated with interstitial cystitis. In some embodiments, the pain associated with bladder pain syndrome is acute and/or chronic pain associated with bladder pain syndrome. In some embodiments, the pain associated with CP/CPPS is acute and/or chronic pain associated with CP/CPPS.

In some embodiments, the pain associated with urologic chronic pelvic pain syndrome (UCPPS) is chronic pain associated with urologic chronic pelvic pain syndrome (UCPPS). In some embodiments, the pain associated with interstitial cystitis/bladder pain syndrome (IC/BPS) is chronic pain associated with interstitial cystitis/bladder pain syndrome (IC/BPS). In some embodiments, the pain associated with interstitial cystitis is chronic pain associated with interstitial cystitis. In some embodiments, the pain associated with bladder pain syndrome is chronic pain associated with bladder pain syndrome. In some embodiments, the pain associated with CP/CPPS is chronic pain associated with CP/CPPS.

In some embodiments, the pain associated with urologic chronic pelvic pain syndrome (UCPPS) is acute pain associated with urologic chronic pelvic pain syndrome (UCPPS). In some embodiments, the pain associated with interstitial cystitis/bladder pain syndrome (IC/BPS) is acute pain associated with interstitial cystitis/bladder pain syndrome (IC/BPS). In some embodiments, the pain associated with interstitial cystitis is acute pain associated with interstitial cystitis. In some embodiments, the pain associated with bladder pain syndrome is acute pain associated with bladder pain syndrome. In some embodiments, the pain associated with CP/CPPS is acute pain associated with CP/CPPS.

In some embodiments, the acute pain associated with UCPPS is a transient exacerbation or flare up of pain associated with UCPPS. In some embodiments, the acute pain associated with interstitial cystitis/bladder pain syndrome (IC/BPS) is a transient exacerbation or flare up of pain associated with interstitial cystitis/bladder pain syndrome (IC/BPS). In some embodiments, the acute pain associated with interstitial cystitis is a transient exacerbation or flare up of pain associated with interstitial cystitis. In some embodiments, the acute pain associated with bladder pain syndrome is a transient exacerbation or flare up of pain associated with bladder pain syndrome. In some embodiments, the acute pain associated with CP/CPPS is a transient exacerbation or flare up of pain associated with CP/CPPS.

In some embodiments, the acute pain is pain that is expected to resolve in a short period of time, for example, 14 days or less. In some embodiments, chronic pain is pain that is expected to persist for an extended period of time, for example, 15 days or longer. Acute pain is distinguishable from chronic pain.

In some embodiments, the acute pain persists for less than about 14 days, less than about 12 days, less than about 10 days, less than about 7 days, less than about 5 days or less than about 3 days from onset of the acute pain. In some embodiments, the acute pain persists for less than about 14 days, less than about 12 days, less than about 10 days from, less than about 7 days, less than about 5 days or less than 3 days from onset of the acute pain. In some embodiments, the acute pain persists for about 1 day to about 14 days, about 1 day to about 12 days, about 1 day to about 10 days, about 1 day to about 7 days, about 1 day to about 5 days, about 1 day to about 3 days, or about 3 days to about 14 days, about 3 days to about 10 days, about 3 days to about 7 days or about 5 days to about 10 days.

In some embodiments, the acute pain occurs intermittently, for more than 3 months, or more than 6 months.

The present application also includes a method of treating urologic chronic pelvic pain syndrome (UCPPS) in a subject in need thereof comprising administering, to the subject, an effective amount of a compound of Formula I,

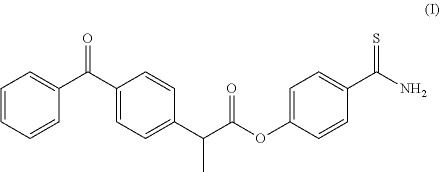 [SMILES: CC(C(=O)Oc1ccc(C(N)=S)cc1)c1ccc(C(=O)c2ccccc2)cc1]

or a pharmaceutically acceptable salt, prodrug and/or solvate thereof.

The present application also includes a use of a compound of Formula I, or a pharmaceutically acceptable salt, prodrug and/or solvate thereof, for treating UCPPS in a subject; a use of a compound of Formula I, or a pharmaceutically acceptable salt, prodrug and/or solvate thereof, for preparation of a medicament for treating UCPPS in a subject; and compound of Formula I, or a pharmaceutically acceptable salt, prodrug and/or solvate thereof, for use to treat UCPPS in a subject.

In some embodiments, the UCPPS includes interstitial cystitis/bladder pain syndrome (IC/BPS) and chronic prostatitis/chronic pelvic pain syndrome (CP/CPPS).

Accordingly, the present application includes a method of treating interstitial cystitis/bladder pain syndrome (IC/BPS) in a subject in need thereof comprising administering, to the subject, an effective amount of a compound of Formula I,

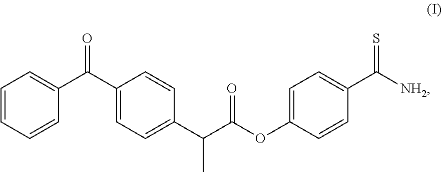 [SMILES: CC(C(=O)Oc1ccc(C(N)=S)cc1)c1ccc(C(=O)c2ccccc2)cc1]

or a pharmaceutically acceptable salt, prodrug and/or solvate thereof.

The present application also includes a use of a compound of Formula I, or a pharmaceutically acceptable salt, prodrug and/or solvate thereof, for treating IC/BPS in a subject; a use of a compound of Formula I, or a pharmaceutically acceptable salt, prodrug and/or solvate thereof, for preparation of a medicament for treating IC/BPS in a subject; and compound of Formula I, or a pharmaceutically acceptable salt, prodrug and/or solvate thereof, for use to treat IC/BPS in a subject.

In some embodiments, the compound of Formula I or a pharmaceutically acceptable salt, prodrug and/or solvate thereof is used or administered for treating interstitial cystitis. In some embodiments, the compound of Formula I or a pharmaceutically acceptable salt, prodrug and/or solvate thereof is used or administered for treating bladder pain syndrome.

The present application also includes a method of treating chronic prostatitis/chronic pelvic pain syndrome (CP/CPPS) in a subject in need thereof comprising administering, to the subject, an effective amount of a compound of

Formula I,

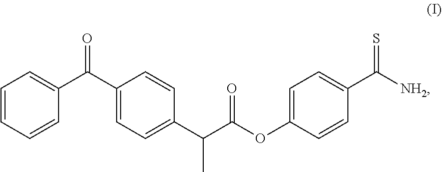 [SMILES: CC(C(=O)Oc1ccc(C(N)=S)cc1)c1ccc(C(=O)c2ccccc2)cc1]

or a pharmaceutically acceptable salt, prodrug and/or solvate thereof.

The present application also includes a use of a compound of Formula I, or a pharmaceutically acceptable salt, prodrug and/or solvate thereof, for treating CP/CPPS in a subject; a use of a compound of Formula I, or a pharmaceutically acceptable salt, prodrug and/or solvate thereof, for preparation of a medicament for treating CP/CPPS in a subject; and compound of Formula I, or a pharmaceutically acceptable salt, prodrug and/or solvate thereof, for use to treat CP/CPPS in a subject.

The present application also includes a method of treating one or more symptoms associated with urologic chronic pelvic pain syndrome (UCPPS) in a subject in need thereof comprising administering, to the subject, an effective amount of a compound of Formula I,

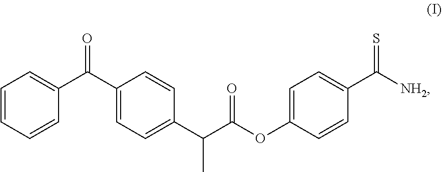 [SMILES: CC(C(=O)Oc1ccc(C(N)=S)cc1)c1ccc(C(=O)c2ccccc2)cc1]

or a pharmaceutically acceptable salt, prodrug and/or solvate thereof.

The present application also includes a use of a compound of Formula I, or a pharmaceutically acceptable salt, prodrug and/or solvate thereof, for treating one or more symptoms associated with UCPPS in a subject; a use of a compound of Formula I, or a pharmaceutically acceptable salt, prodrug and/or solvate thereof, for preparation of a medicament for treating one or more symptoms associated with UCPPS in a subject; and compound of Formula I, or a pharmaceutically acceptable salt, prodrug and/or solvate thereof, for use to treat treating one or more symptoms associated with UCPPS in a subject.

In some embodiments, the UCPPS includes IC/BPS and/or CP/CPPS. Therefore, in some embodiments, the application also includes a method or use of the compound of Formula I, or a pharmaceutically acceptable salt, prodrug and/or solvate thereof, for treating one or more symptoms associated with IC/BPS. In some embodiments, the application also includes a method or use of the compound of Formula I, or a pharmaceutically acceptable salt, prodrug and/or solvate thereof, for treating one or more symptoms associated with CP/CPPS.

In some embodiments, the one or more symptoms are selected from irritative voiding symptoms, urinary frequency, urinary urgency, nocturia, incontinence and suprapubic or pelvic pain related to and relieved by voiding.

The Applicants have shown that the compound of Formula I can be used to alleviate bladder inflammation. Therefore, the present application also includes a method of treating bladder inflammation in a subject in need thereof comprising administering, to the subject, an effective amount of a compound of Formula I,

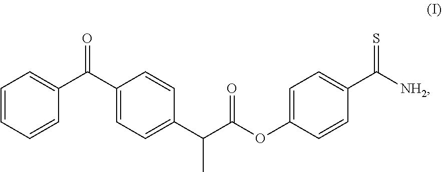 [SMILES: CC(C(=O)Oc1ccc(C(N)=S)cc1)c1ccc(C(=O)c2ccccc2)cc1]

or a pharmaceutically acceptable salt, prodrug and/or solvate thereof.

The present application also includes a use of a compound of Formula I, or a pharmaceutically acceptable salt, prodrug and/or solvate thereof, for treating bladder inflammation in a subject; a use of a compound of Formula I, or a pharmaceutically acceptable salt, prodrug and/or solvate thereof, for preparation of a medicament for treating bladder inflammation in a subject; and compound of Formula I, or a pharmaceutically acceptable salt, prodrug and/or solvate thereof, for use to treat bladder inflammation in a subject.

In some embodiments, the bladder inflammation is associated with urologic chronic pelvic pain syndrome (UCPPS). In some embodiments, the bladder inflammation is associated with interstitial cystitis/bladder pain syndrome (IC/BPS). In some embodiments, the bladder inflammation is associated with interstitial cystitis. In some embodiments, the bladder inflammation is associated with bladder pain syndrome. In some embodiments, the bladder inflammation is associated with chronic prostatitis/chronic pelvic pain syndrome (CP/CPPS).

The present application includes a method of increasing urinary bladder contraction and relaxation in a subject in need thereof comprising administering, to the subject, an effective amount of a compound of Formula I,

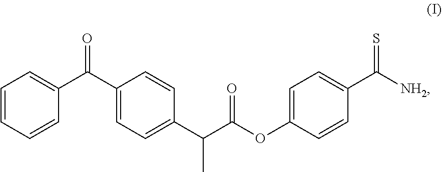 [SMILES: CC(C(=O)Oc1ccc(C(N)=S)cc1)c1ccc(C(=O)c2ccccc2)cc1]

or a pharmaceutically acceptable salt, prodrug and/or solvate thereof.

The present application also includes a use of a compound of Formula I, or a pharmaceutically acceptable salt, prodrug and/or solvate thereof, for increasing urinary bladder contraction and relaxation in a subject; a use of a compound of Formula I, or a pharmaceutically acceptable salt, prodrug and/or solvate thereof, for preparation of a medicament for increasing urinary bladder contraction and relaxation in a subject; and compound of Formula I, or a pharmaceutically acceptable salt, prodrug and/or solvate thereof, for use to treat increasing urinary bladder contraction and relaxation in a subject.

In some embodiments, the subject is a subject having UCPPS. In some embodiments, the UCPPS is IC/BPS and/or CP/CPPS.

The present application includes a method of treating acute pain in a subject in need thereof comprising administering, to the subject, an effective amount of a compound of Formula I,

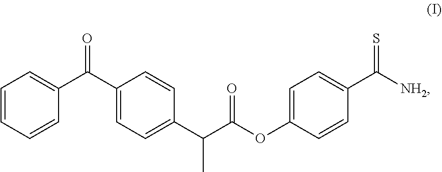 [SMILES: CC(C(=O)Oc1ccc(C(N)=S)cc1)c1ccc(C(=O)c2ccccc2)cc1]

or a pharmaceutically acceptable salt, prodrug and/or solvate thereof.

The present application also includes a use of a compound of Formula I, or a pharmaceutically acceptable salt, prodrug and/or solvate thereof, for treating acute pain in a subject; a use of a compound of Formula I, or a pharmaceutically acceptable salt, prodrug and/or solvate thereof, for preparation of a medicament for treating acute pain in a subject; and compound of Formula I, or a pharmaceutically acceptable salt, prodrug and/or solvate thereof, for use to treat acute pain in a subject.

In some embodiments, the acute pain is a transient exacerbation or flare up.

In some embodiments, the acute pain is pain that is expected to resolve in a short period of time, for example, 14 days or less. In some embodiments, the acute pain persists for less than about 14 days, less than about 12 days, less than about 10 days, less than about 7 days, less than about 5 days or less than about 3 days from onset of the acute pain. In some embodiments, the acute pain persists for less than about 14 days, less than about 12 days, less than about 10 days from, less than about 7 days, less than about 5 days or less than 3 days from onset of the acute pain. In some embodiments, the acute pain persists for about 1 day to about 14 days, about 1 day to about 12 days, about 1 day to about 10 days, about 1 day to about 7 days, about 1 day to about 5 days, about 1 day to about 3 days, or about 3 days to about 14 days, about 3 days to about 10 days, about 3 days to about 7 days or about 5 days to about 10 days.

In some embodiments, the acute pain occurs intermittently, for more than 3 months, or more than 6 months.

In some embodiments, the acute pain is post-operative pain resulting from abdominal and/or pelvic surgery. The present application further includes a method of treating acute pain resulting from abdominal and/or pelvic surgery in a subject in need thereof comprising administering, to the subject, an effective amount of a compound of Formula I,

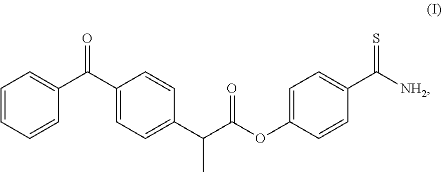 [SMILES: CC(C(=O)Oc1ccc(C(N)=S)cc1)c1ccc(C(=O)c2ccccc2)cc1]

or a pharmaceutically acceptable salt, prodrug and/or solvate thereof.

The present application also includes a use of a compound of Formula I, or a pharmaceutically acceptable salt, prodrug and/or solvate thereof, for treating acute pain resulting from abdominal and/or pelvic surgery in a subject; a use of a compound of Formula I, or a pharmaceutically acceptable salt, prodrug and/or solvate thereof, for preparation of a medicament for treating acute pain resulting from abdominal and/or pelvic surgery in a subject; and compound of Formula I, or a pharmaceutically acceptable salt, prodrug and/or solvate thereof, for use to treat acute pain resulting from abdominal and/or pelvic surgery in a subject.

In some embodiments, the abdominal and/or pelvic surgery is selected from, but not limited to, inguinal hernia repair such as open inguinal hernectomy, abdominal hysterectomy, abdominal laparotomy, cholecystectomy, vaginal hysterectomy, ventral hernia repair, myomectomy, salpingo-oophorectomy, bariatric, partial colectomy surgeries, and gynecologic or genitourinary surgery.

In some embodiments, the abdominal and/or pelvic surgery is selected from, but not limited to, inguinal hernia repair such as open inguinal hernectomy, abdominal hysterectomy, abdominal laparotomy or bariatric surgery. In some embodiments, the abdominal and/or pelvic surgery is inguinal hernia repair. In some embodiments, inguinal hernia repair is open inguinal herniotomy.

In some embodiments, the abdominal and/or pelvic surgery is abdominal laparotomy or bariatric surgery. In some embodiments, the abdominal and/or pelvic surgery is bariatric surgery.

In some embodiments, the acute pain is not post-operative pain.

In some embodiments, the subject in need thereof is a subject having urologic chronic pelvic pain syndrome (UCPPS), pain associated with UCPPS and/or bladder inflammation. In some embodiments, the subject in need thereof is a subject having interstitial cystitis/bladder pain syndrome (IC/BPS), pain associated with IC/BPS and/or bladder inflammation. In some embodiments, the subject in need thereof is a subject having interstitial cystitis, pain associated with interstitial cystitis and/or bladder inflammation. In some embodiments, the subject in need thereof is a subject having bladder pain syndrome, pain associated with bladder pain syndrome and/or bladder inflammation. In some embodiments, the subject in need thereof is a subject having chronic prostatitis/chronic pelvic pain syndrome (CP/CPPS), pain associated with CP/CPPS and/or bladder inflammation.

In an embodiment, the compound of Formula I is in the form of a pharmaceutically acceptable solvate. In an embodiment, the compound of Formula I is in a neutral form (i.e., not a salt). In an embodiment, the compound of Formula I is a prodrug of the compound of Formula I. In an embodiment, the compound of Formula I is a solvate of a prodrug of the compound of Formula I.

The present application also includes a method of treating urologic chronic pelvic pain syndrome (UCPPS), pain associated with UCPPS, bladder inflammation and/ or acute pain by administering an effective amount of a compound of Formula I, or a pharmaceutically acceptable salt, prodrug and/or solvate thereof, to a subject in need thereof in combination with another known agent useful for treating urologic chronic pelvic pain syndrome (UCPPS), pain associated with UCPPS, bladder inflammation and/ or acute pain. The present application also includes a use of a compound of Formula I or a pharmaceutically acceptable salt, prodrug and/or solvate thereof, for treating urologic chronic pelvic pain syndrome (UCPPS), pain associated with UCPPS, bladder inflammation and/or acute pain in a subject in need thereof in combination with another known agent useful for treating urologic chronic pelvic pain syndrome (UCPPS), pain associated with UCPPS, bladder inflammation and/or acute pain. The present application also includes a use of a compound of Formula I or a pharmaceutically acceptable salt, prodrug and/or solvate thereof, for preparing a medicament for treating urologic chronic pelvic pain syndrome (UCPPS), pain associated with UCPPS, bladder inflammation and/or acute pain in combination with another known agent useful for treating urologic chronic pelvic pain syndrome (UCPPS), pain associated with UCPPS, bladder inflammation and/or acute pain in a subject in need thereof. The application further includes compound of Formula I or a pharmaceutically acceptable salt, prodrug and/or solvate thereof, for use to treat urologic chronic pelvic pain syndrome (UCPPS), pain associated with UCPPS, bladder inflammation and/or acute pain in a subject in need thereof in combination with another known agent useful for treating urologic chronic pelvic pain syndrome (UCPPS), pain associated with UCPPS, bladder inflammation and/or acute pain. In some embodiments, the method or use is for treating pain associated with UCPPS. In some embodiments, the method or use is for treating pain associated with IC/BPS. In some embodiments, the method or use is for treating pain associated with interstitial cystitis. In some embodiments, the method or use is for treating pain associated with bladder pain syndrome. In some embodiments, the method or use is for treating pain associated with CP/CPPS. In some embodiments, the pain is acute pain. In some embodiments, the method or use is for treating IC/BPS. In some embodiments, the method or use is for treating interstitial cystitis. In some embodiments, the method or use is for treating bladder pain syndrome. In some embodiments, the method or use is for treating with CP/CPPS.

In some embodiments, when used with other known agents useful for treating or preventing pain associated with UCPPS, UCPPS or bladder inflammation, the methods and uses of the application reduce the amount of the other known agents used or administered. In some embodiments, when used with other known agents useful for treating or preventing pain associated with UCPPS, UCPPS or bladder inflammation, the methods and uses of the application reduce the amount of the other known agents by about 40% to 60% within about 12 hours, about 24 hours, about 36 hours, about 48 hours, about 60 hours or about 72 hours post administration.

In some embodiments, the another known agent for treating UCPPS or pain associated with UCPPS is selected from antihistamines including H1 blockers such as hydroxyzine hydrochloride and H2 blockers such as cimetidine, dimethylsulfoxide, pain modulations including tricyclic antidepressants such as amitriptylin, anticonvulsants such as gabapentin, and dimethylsulfoxide, narcotic antagonists, corticosteroids, antirheumatics, immunoregulators, immunosuppressors such as prednisone and triamcinolone, interleukin production inhibitors, bladder mucosal protectors such as pentosan polysulphate and hyaluronic acid, chondroitin sulphate leukotriene-D4 receptor antagonist montelukast, L-arginine, oxybutynin, tolterodine, hormone modulators such as leuprolide acetate, anti-inflammatory agents such as anti-TNF, narcotics and pain relief agents such as opioids and tramadol.

In an embodiment, the corticosteroid is selected from cortisone, hydrocortisone, prednisone, methylprednisolone, deflazacort, prednisolone, triamcinolone, dexamethasone, betamethasone, halopredone, beclomethasone and fludrocortisone and a combination thereof.

In some embodiments, the compound of Formula I or a pharmaceutically acceptable salt and/or solvate thereof is used or administered alone without other known agents useful for treating or preventing pain associated with urologic chronic pelvic pain syndrome (UCPPS).

It would be appreciated by a person skilled in the art that pain rating scales are well known and used in daily clinical practice to measure pain intensity. For example, commonly used measurement scales include the Visual Analog Scale (VAS), the Graphic Rating Scale (GRS), the Simple Descriptor Scale (SDS), the Numerical Rating Scale (NRS), the Faces Rating Scale (FRS) and Total Pain Relief (TOTPAR) and/or Sum of Pain Intensity Difference (SPID). Particularly in acute pain, Total Pain Relief (TOTPAR) and/or Sum of Pain Intensity Difference (SPID) are the metrics commonly used to assess pain in the art. Numeric Rating Scales (NRS) which are similar to VAS in acute pain trials are also available. In some embodiments, the visual analog scale (VAS) is a 10 cm. vertical or horizontal line with word anchors at the extremes, such as “no pain” on one end and “pain as bad as it could be” at the other. The patient is asked to make a mark along the line to represent pain intensity. In the Visual Analog Scale (VAS), moderate or severe pain is defined as a pain intensity of >about 50 mm on a 0-100 mm visual analog scale (VAS). In some embodiments, moderate pain is characterized as a pain intensity of >about 50 mm and <about 70 mm on a 1-100 mm VAS. And severe pain is characterized as a pain intensity of >about 70 mm on a 1-100 mm VAS. The graphic rating scale (GRS) is a variation of the visual scale which adds words such as include “no pain”, “mild”, “severe or numbers between the extremes. The descriptor scale (SDS) is a list of adjectives such as no pain”, “mild”, “moderate” or “severe describing different levels of pain intensity. The numerical pain rating scale (NPRS) refers to a numerical rating of 0 to 10 or 0 to 5 or to a visual scale with both words and numbers. A visual analogue scale (VAS) version of the WOMAC is also commonly used.

In some embodiments, treatment of the subject is assessed using one or more biomarkers known to represent activation of pathways that result in pain reduction, including but not limited to, plasma levels of thromboxane B₂ (TXB₂), prostaglandin E₂ (PGE2), interleukin 1 Beta (IL-1β), interleukin 6 (IL-6), tumor necrosis factor alpha (TNF-α) and/or C-reactive protein (CRP). Levels of these markers that are indicative of a pain response are known in the art as are methods to detect their levels in a subject.

In some embodiments, the pain is mild to moderate, moderate to moderately severe or moderately severe to severe.

In some embodiments, the methods and uses of the application provide pain relief within about 30 minutes to about 4 hours, about 1 hour to about 4 hours, about 1 hour to about 3 hours, about 1 hour to about 3 hours, about 1 hour to about 2 hours, about 2 hours to about 3 hours after administration of the compound of Formula I. In some embodiments, the methods of the application provide pain relief within about 1 hour to about 3 hours, about 1 hour to about 2 hours, about 2 hours to about 3 hours after administration of the compound of Formula I.

Effective amounts may vary according to factors such as the disease state, age, sex and/or weight of the subject. The amount of a given compound that will correspond to such an amount will vary depending upon various factors, such as the given drug or compound, the pharmaceutical formulation, the route of administration, the type of viral infection of the subject being treated, and the like, but can nevertheless be routinely determined by one skilled in the art. The effective amount is one that following treatment therewith manifests as an improvement in or reduction of any disease symptom.

The dosage of the compound of Formula I or a pharmaceutically acceptable salt, prodrug and/or solvate thereof can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. The compound of Formula I or a pharmaceutically acceptable salt, prodrug and/or solvate thereof may be administered initially in a suitable dosage that may be adjusted as required, depending on the clinical response. Dosages will generally be selected to maintain a serum level of compounds from about 0.01 μg/cc to about 1000 μg/cc, or about 0.1 μg/cc to about 100 μg/cc. As a representative example, oral dosages of the compound of Formula I or a pharmaceutically acceptable salt, prodrug and/or solvate thereof will range between about 1 mg per day to about 1000 mg per day for an adult, suitably about 50 mg, about 100 mg, about 150 mg, about 200 mg, about 250 mg, about 300 mg, about 350 mg, about 400 mg, about 450 mg or about 500 mg per day. For parenteral administration or use, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered or used. For oral administration or use, a representative amount is from about 0.001 mg/kg to about 10 mg/kg or about 0.1 mg/kg to about 10 mg/kg. For administration or use in suppository form, a representative amount is from about 0.1 mg/kg to about 10 mg/kg.

In some embodiments, oral dosages of the compound of Formula I or a pharmaceutically acceptable salt, prodrug and/or solvate thereof will range from about 50 mg per day to about 500 mg per day for an adult, suitably about 50 mg, about 100 mg, about 150 mg, about 200 mg, about 250 mg, about 275 mg, about 300 mg, about 325 mg, about 350 mg, about 375 mg, about 400 mg, about 425 mg, about 450 mg, about 475 mg or about 500 mg per day. In some embodiments, oral dosages of the compound of Formula I or a pharmaceutically acceptable salt, prodrug and/or solvate thereof will range from 50 mg per day to about 500 mg per day for an adult, suitably about 50 mg, about 100 mg, about 150 mg, about 200 mg, about 250 mg, about 275 mg, about 300 mg, about 325 mg, about 350 mg, about 375 mg, about 400 mg, about 425 mg, about 450 mg, about 475 mg or about 500 mg per day. In some embodiments, the oral dosages of the compound of Formula I or a pharmaceutically acceptable salt, prodrug and/or solvate thereof will range from about 50 mg per day to about 500 mg per day, about 100 mg per day to about 400 mg per day, about 200 mg per day to about 400 mg per day, about 250 mg per day to about 350 mg per day or about 250 mg per day to about 325 mg per day In some embodiments, the oral dosages of the compound of Formula I or a pharmaceutically acceptable salt, prodrug and/or solvate thereof will range from about 200 mg per day to about 400 mg per day or about 250 mg per day to about 325 mg per day. In some embodiments, the oral dosage of the compound of Formula I or a pharmaceutically acceptable salt, prodrug and/or solvate thereof is about 325 mg per day.

In some embodiments, the compound of Formula I is formulated for oral administration or use and the compound of Formula I is suitably in the form of tablets or other solid form oral dosage form containing 0.25, 0.5, 0.75, 1, 5, 10, 20, 25, 30, 40, 50, 60, 70, 75, 80, 90, 100, 125, 150, 175, 200, 225, 250, 275, 300, 325, 350, 400, 450 or 500 mg of active ingredient per tablet.

In some embodiments, the compound of Formula I or a pharmaceutically acceptable salt, prodrug and/or solvate thereof may be administered or used in a single daily dose or the total daily dose may be divided into two, three, four, five or six daily doses. In some embodiments, the compound of Formula I or a pharmaceutically acceptable salt, prodrug and/or solvate thereof is administered 2, 3, 4, 5 or 6 times daily.

In some embodiments, for example, when the compound of Formula I is used or administered to treat acute pain including acute pain associated with UCPPS the compound of Formula I or a pharmaceutically acceptable salt, prodrug and/or solvate thereof is administered for 1 day to 14 days, for 2 days to 10 days, for 2 days to 9 days, for 2 days to 8 days, for 2 days to 7 days, for 2 days to 6 days, or for 3 days to 5 days. In some embodiments, the acute pain is a transient exacerbation or flare up. In some embodiments, the compound of Formula I is used or administered as described intermittently, for more than 3 months, or more than 6 months.

In some embodiments, the oral dosage of the compound of Formula I or a pharmaceutically acceptable salt, prodrug and/or solvate thereof is about 400 mg to about 500 mg per day and the compound of Formula I or a pharmaceutically acceptable salt, prodrug and/or solvate thereof is administered for 1 day. In some embodiments, the oral dosage of the compound of Formula I or a pharmaceutically acceptable salt, prodrug and/or solvate thereof is about 400 mg to about 500 mg per day, about 400 mg to about 450 mg per day or about 450 mg to about 500 mg per day or about 400 mg per day, about 450 mg per day or about 500 mg per day and the compound of Formula I or a pharmaceutically acceptable salt, prodrug and/or solvate thereof is administered for 1 day.

In some embodiments, the oral dosage of the compound of Formula I or a pharmaceutically acceptable salt, prodrug and/or solvate thereof is about 200 mg per day to about 400 mg per day, about 250 mg per day to about 350 mg per day or about 250 mg per day to about 325 mg per day and the compound of Formula I or a pharmaceutically acceptable salt, prodrug and/or solvate thereof is administered for 1 day to 10 days, 1 day to 8 days, 1 day to 7 days, 1 day to 5 days, 2 days to 7 days, 3 days to 7 days, 4 days to 7 days, 5 days to 7 days, 6 days to 7 days, 2 days to 5 days, 3 days to 5 days, 4 days to 5 days, 2 days, 3 days, 4 days, 5 days, 6 days or 7 days. In some embodiments, the oral dosages of the compound of Formula I or a pharmaceutically acceptable salt, prodrug and/or solvate thereof is about 250 mg per day to about 350 mg per day or about 250 mg per day to about 325 mg per day, or about 250 mg per day, about 275 mg per day, about 300 mg per day, about 325 mg per day or about 350 mg per day, and the compound of Formula I or a pharmaceutically acceptable salt, prodrug and/or solvate thereof is administered for 1 day to 8 days, 1 day to 7 days, 1 day to 5 days, 2 days to 7 days, 3 days to 7 days, 4 days to 7 days, 5 days to 7 days, 6 days to 7 days, 2 days to 5 days, 3 days to 5 days, 4 days to 5 days, 2 days, 3 days, 4 days, 5 days, 6 days or 7 days.

The length of the treatment period depends on a variety of factors, such as the severity of the acute pain, the age of the subject, the concentration and/or the activity of the compound of Formula I or a pharmaceutically acceptable salt, prodrug and/or solvate, and/or a combination thereof. It will also be appreciated that the effective dosage of the compound used for the treatment may increase or decrease over the course of a particular treatment regime. Changes in dosage may result and become apparent by standard diagnostic assays known in the art. For example, the compound of Formula I or a pharmaceutically acceptable salt, prodrug and/or solvate thereof is administered to the subject in an amount and for duration sufficient to treat the subject.

In an embodiment, the subject is a mammal. In another embodiment, the subject is human. In an embodiment, the subject is a non-human animal.

The compound of Formula I, or a pharmaceutically acceptable salt, prodrug and/or solvate thereof is either used or administered alone or in combination with other known agents useful for treating pain associated with urologic chronic pelvic pain syndrome (UCPPS), UCPPS, acute pain and/or bladder inflammation. When used or administered in combination with other agents or therapies, it is an embodiment that compound of Formula I, or a pharmaceutically acceptable salt, prodrug and/or solvate thereof, is used or administered contemporaneously with those agents or therapies. As used herein, “contemporaneous administration” or “contemporaneous use” of two substances or therapies to a subject means providing each of the two substances or therapies so that they are both biologically active in the individual at the same time. The exact details of the administration or use will depend on the pharmacokinetics of the two substances or therapies in the presence of each other, and can include administering or using the two substances or therapies within a few hours of each other, or even administering or using one substance or therapy within 24 hours of administration or use of the other, if the pharmacokinetics are suitable. Design of suitable dosing regimens is routine for one skilled in the art. In particular embodiments, the substances or therapies will be administered or used substantially simultaneously, i.e., within minutes of each other, or in a single composition in the case of administration or use of two or more substances. It is a further embodiment of the present application that a combination of agents or therapies is administered to a subject or used in a non-contemporaneous fashion.

In some embodiments, the compound of Formula I or a pharmaceutically acceptable salt and/or solvate thereof or a composition of the application is used or administered in combination with other known agents useful for treating or preventing pain associated with urologic chronic pelvic pain syndrome (UCPPS), UCPPS, acute pain and/or bladder inflammation.

In an embodiment of the methods and uses of the application, the compound of Formula I or a pharmaceutically acceptable salt, prodrug and/or solvate thereof is administered or used orally. In an embodiment, the compound of Formula I or a pharmaceutically acceptable salt, prodrug and/or solvate thereof is administered or used by parenteral administration. In an embodiment, the compound of Formula I or a pharmaceutically acceptable salt, prodrug and/or solvate thereof is administered or used by intramuscular, transepithelial, topical including transdermal administration.

In an embodiment the pharmaceutically acceptable salt is an acid addition salt or a base addition salt. The selection of a suitable salt may be made by a person skilled in the art (see, for example, S. M. Berge, et al., “Pharmaceutical Salts,” J. Pharm. Sci. 1977, 66, 1-19).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylam inoethanol, 2-diethylam inoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolyzed. The selection criteria for the appropriate salt will be known to one skilled in the art.

Solvates of compound of Formula I include, for example, those made with solvents that are pharmaceutically acceptable. Examples of such solvents include water (resulting solvate is called a hydrate) and ethanol and the like. Suitable solvents are physiologically tolerable at the dosage administered.

The compound of Formula I or a pharmaceutically acceptable salt, prodrug and/or solvate thereof may further exist in an amorphous form or varying polymorphic forms and it is contemplated that any amorphous form, any polymorphs, or mixtures thereof, which form are included within the scope of the present application.

The compound of Formula I or a pharmaceutically acceptable salt, prodrug and/or solvate thereof may further be radiolabeled and accordingly all radiolabeled versions of the compounds of the application are included within the scope of the present application. The compounds of the application also include those in which one or more radioactive atoms are incorporated within their structure.

### III. Compositions of the Application

In an embodiment, the compound of Formula I or a pharmaceutically acceptable salt, prodrug and/or solvate thereof

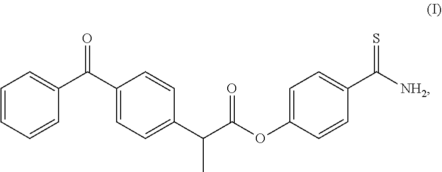 [SMILES: CC(C(=O)Oc1ccc(C(N)=S)cc1)c1ccc(C(=O)c2ccccc2)cc1]

is suitably formulated in a conventional manner into compositions using one or more carriers.

The compound of Formula I or a or a pharmaceutically acceptable salt and/or solvate thereof is suitably formulated into pharmaceutical compositions for administration to subjects in a biologically compatible form suitable for administration in vivo. Accordingly, the compound of Formula I or a pharmaceutically acceptable salt and/or solvate thereof are suitably formulated into pharmaceutical compositions comprising a compound of Formula I or a pharmaceutically acceptable salt, prodrug and/or solvate thereof

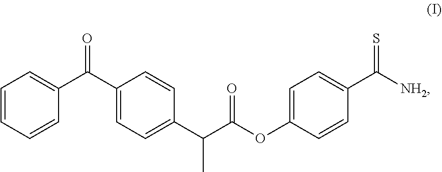 [SMILES: CC(C(=O)Oc1ccc(C(N)=S)cc1)c1ccc(C(=O)c2ccccc2)cc1]

and a pharmaceutically acceptable carrier, respectively.

Therefore, the present application further includes a method or use for treating pain associated with urologic chronic pelvic pain syndrome (UCPPS), UCPPS, acute pain and/or bladder inflammation as described above by administering or using a composition comprising a compound of Formula I or a pharmaceutically acceptable salt, prodrug and/or solvate thereof.

The compound of Formula I or a pharmaceutically acceptable salt, prodrug and/or solvate thereof is suitably used on its own but will generally be administered in the form of a composition in association with an acceptable carrier. Depending on the mode of administration, the composition will comprise from about 0.05 wt % to about 99 wt % or about 0.10 wt % to about 70 wt %, of the one or more forms, and from about 1 wt % to about 99.95 wt % or about 30 wt % to about 99.90 wt % of an acceptable carrier, all percentages by weight being based on the total composition

The compound of Formula I or a pharmaceutically acceptable salt, prodrug and/or solvate thereof, are also administered to a subject, or used, in a variety of forms depending on the selected route of administration, as will be understood by those skilled in the art. In an embodiment, the compound of Formula I or a pharmaceutically acceptable salt, prodrug and/or solvate thereof are administered to the subject, or used, by oral (including sublingual and buccal) or parenteral (including, intravenous, intraperitoneal, subcutaneous, intramuscular, transepithelial, nasal, intrapulmonary, intrathecal, rectal, topical, patch, pump and transdermal) administration and the compound, salt, prodrug and/or solvate, formulated accordingly. Again, conventional procedures and ingredients for the selection and preparation of suitable compositions are described, for example, in Remington's Pharmaceutical Sciences (2000—20th edition) and in The United States Pharmacopeia: The National Formulary (USP 24 NF19) published in 1999.

Parenteral administration includes intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time. The pharmaceutical forms suitable for injectable use include sterile aqueous solutions or dispersions and sterile powders for the extemporaneous preparation of sterile injectable solutions or dispersions. In all cases, the form is sterile and fluid to the extent that easy syringability exists.

The compound of Formula I or a pharmaceutically acceptable salt, prodrug and/or solvate thereof may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound may be incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions can be formulated, e.g., liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. Liposomes can be formed from a variety of phospholipids, such as cholesterol, stearylamine or phosphatidylcholines. For oral administration in a capsule form, useful carriers or diluents include lactose and dried corn starch.

Liquid preparations for oral administration may take the form of, for example, solutions, syrups or suspensions, or they are suitably presented as a dry product for constitution with water or other suitable vehicle before use. When aqueous suspensions and/or emulsions are administered orally, the compound of Formula I or a pharmaceutically acceptable salt, prodrug and/or solvate thereof is suitably suspended or dissolved in an oily phase that is combined with emulsifying and/or suspending agents. If desired, certain sweetening and/or flavoring and/or coloring agents may be added. Such liquid preparations for oral administration may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compound of Formula I or a pharmaceutically acceptable salt, prodrug and/or solvate thereof and use the lyophilizates obtained, for example, for the preparation of products for injection.

The compound of Formula I or a pharmaceutically acceptable salt, prodrug and/or solvate thereof may also be administered parenterally. Solutions of the compound of Formula I or a pharmaceutically acceptable salt, prodrug and/or solvate thereof can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compound of Formula I or a pharmaceutically acceptable salt, prodrug and/or solvate thereof are usually prepared, and the pH of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

The compound of Formula I or a pharmaceutically acceptable salt, prodrug and/or solvate thereof may be formulated for parenteral administration by injection, including using conventional catheterization techniques or infusion. Formulations for injection may be presented in unit dosage form, e.g., in ampoules or in multi-dose containers, with an added preservative. The compositions may take such forms as sterile suspensions, solutions or emulsions in oily or aqueous vehicles, and may contain formulating agents such as suspending, stabilizing and/or dispersing agents. In all cases, the form must be sterile and must be fluid to the extent that easy syringability exists. Alternatively, the compound of Formula I or a pharmaceutically acceptable salt, prodrug and/or solvate thereof are suitably in a sterile powder form for reconstitution with a suitable vehicle, e.g., sterile pyrogen-free water, before use.

Compositions for nasal administration may conveniently be formulated as aerosols, drops, gels and powders.

For intranasal administration or administration by inhalation, the compound of Formula I or a pharmaceutically acceptable salt, prodrug and/or solvate thereof is conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of the compound of Formula I or a pharmaceutically acceptable salt, prodrug and/or solvate thereof and a suitable powder base such as lactose or starch. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

Suppository forms of the compound of Formula I or a pharmaceutically acceptable salt, prodrug and/or solvate thereof is useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, PA, 1980, pp. 1530-1533 for further discussion of suppository dosage forms.

The compound of Formula I or a pharmaceutically acceptable salt, prodrug and/or solvate thereof may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, the compound of Formula I or a pharmaceutically acceptable salt, prodrug and/or solvate thereof may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

In an embodiment, the compound of Formula I or a pharmaceutically acceptable salt, prodrug and/or solvate thereof may be coupled with viral, non-viral or other vectors. Viral vectors may include retrovirus, lentivirus, adenovirus, herpesvirus, poxvirus, alphavirus, vaccinia virus or adeno-associated viruses. Non-viral vectors may include nanoparticles, cationic lipids, cationic polymers, metallic nanoparticles, nanorods, liposomes, micelles, microbubbles, cell-penetrating peptides, or lipospheres. Nanoparticles may include silica, lipid, carbohydrate, or other pharmaceutically acceptable polymers.

In an embodiment, depending on the mode of administration, the pharmaceutical composition will comprise from about 0.05 wt % to about 99 wt % or about 0.10 wt % to about 70 wt %, of the active ingredient (compound of Formula I), and from about 1 wt % to about 99.95 wt % or about 30 wt % to about 99.90 wt % of one or more pharmaceutically acceptable carriers, all percentages by weight being based on the total composition.

In an embodiment, the pharmaceutical composition is formulated for oral administration. In an embodiment, the pharmaceutical composition is formulated for oral administration and comprises about 50 mg, about 100 mg, about 150 mg, about 200 mg, about 250 mg, about 300 mg, about 350 mg, about 400 mg, about 450 mg or about 500 mg of the compound of Formula I or a pharmaceutically acceptable salt, prodrug and/or solvate thereof.

In an embodiment, the compound of Formula I or a pharmaceutically acceptable salt, prodrug and/or solvate thereof is administered with another therapeutic agent simultaneously or sequentially in separate unit dosage forms or together in a single unit dosage form. Accordingly, the present application provides a single unit dosage form comprising a compound of Formula I or a pharmaceutically acceptable salt, prodrug and/or solvate thereof, an additional therapeutic agent, and a pharmaceutically acceptable carrier.

In embodiment, the composition is formulated for parenteral administration. In an embodiment, the composition is formulated for transdermal administration.

The compound of Formula I or a pharmaceutically acceptable salt, prodrug and/or solvate thereof may be used alone to treat acute pain, urologic chronic pelvic pain syndrome (UCPPS) or bladder inflammation or in combination with another known agent useful for treating treat acute pain, urologic chronic pelvic pain syndrome (UCPPS) or bladder inflammation.

It is an embodiment that another known agent useful for treating acute pain, urologic chronic pelvic pain syndrome (UCPPS) or bladder inflammation are administered or used according to treatment protocol that is known the other known agents.

In an embodiment, the compound of Formula I is in the form of a pharmaceutically acceptable solvate. In an embodiment, the compound of Formula I is in a neutral form (i.e., not a salt). In an embodiment, the compound of Formula I is a prodrug of the compound of Formula I. In an embodiment, the compound of Formula I is a solvate of a prodrug of the compound of Formula I.

### IV. Methods of Preparation

The compound of Formula I can be prepared by various synthetic processes. The selection of a particular process is within the purview of the person of skill in the art. Some starting materials are available from commercial chemical sources. Other starting materials, for example as described below, are readily prepared from available precursors using straightforward transformations that are well known in the art.

The compound of Formula I generally can be prepared according to the processes illustrated in the Scheme below. A person skilled in the art would appreciate that many of the reactions depicted in the Scheme below would be sensitive to oxygen and water and would know to perform the reaction under an anhydrous, inert atmosphere if needed. Reaction temperatures and times are presented for illustrative purposes only and may be varied to optimize yield as would be understood by a person skilled in the art.

Accordingly, the compound of Formula I is prepared as shown in Scheme 1. Therefore, the compound of Formula A (ketoprofen) is coupled with the compound of Formula B (hydroxybenzamide) with suitable coupling agents such as dicyclohexylcarbodiimide (DCC) and hydroxybenzotriazole (HOBt) under suitable coupling conditions in a suitable solvent such as dimethylformamide to provide a compound of Formula C. The compound of Formula C is then thionated using a suitable thionating agent such as Lawesson reagent in a suitable solvent such as anhydrous benzene to provide the compound of Formula I.

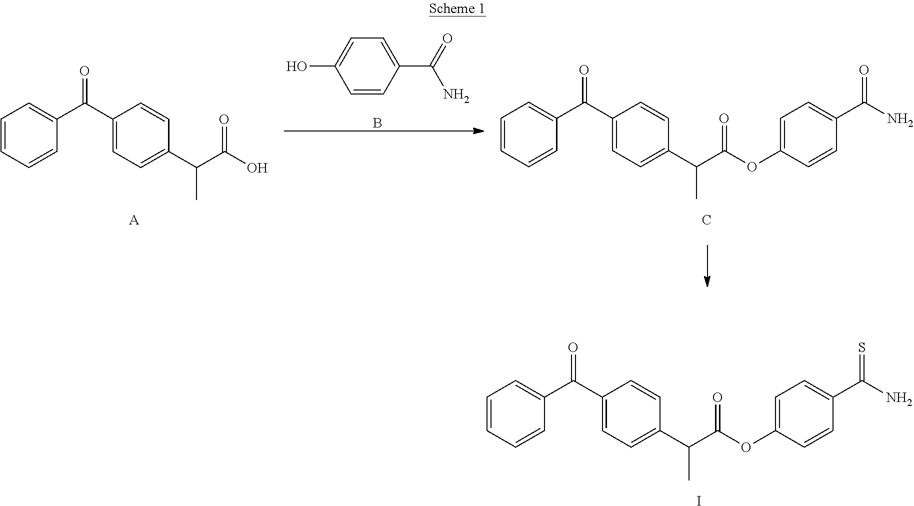

The compound of Formula I, can also be prepared by methods known in the art, for example, by the methods disclosed in U.S. Pat. No. 8,541,398 and/or U.S. Pat. No. 7,910,568.

### EXAMPLES

The following non-limiting examples are illustrative of the present application:

### Example 1: Synthesis of compound of Formula I

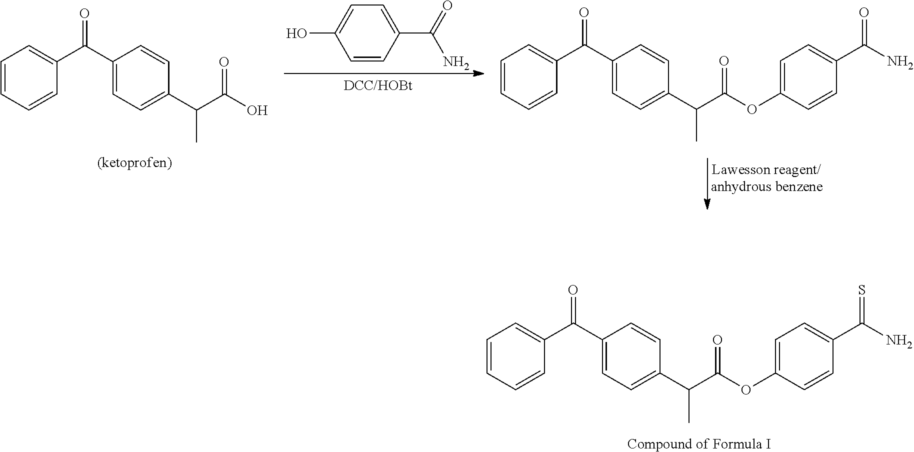 [SMILES: CC(C(=O)O)c1ccc(C(=O)c2ccccc2)cc1 ; NC(=O)c1ccc(O)cc1 ; CC(C(=O)Oc1ccc(C(N)=O)cc1)c1ccc(C(=O)c2ccccc2)cc1 ; "(ketoprofen)" ; CC(C(=O)Oc1ccc(C(N)=S)cc1)c1ccc(C(=O)c2ccccc2)cc1 ; "Compound of Formula I"]

Step 1: Synthesis of 4-carbamoylphenyl 2-(4-oxophenyl)-phenyl Propanoate

To the solution of 1 (ketoprofen, 3 g, 11.8 mmol) in 80 mL of dimethylformamide, hydroxybenzotriazole (1.76 g, 13 mmol) and DCC (2.68 g, 13 mmol) were added with stirring at 0° C. for 1 h. To the reaction mixture 4-hydroxybenzamide (2, 2.43 g, 17.7 mmol) was added and stirred for 1 h at 0° C. and 2 h at room temperature. After filtration, the filtrate was evaporated under reduced pressure to remove the solvent. The oily residue thus obtained was dissolved in ethyl acetate; the organic layer was washed with brine, with NaHCO3 5%, with citric acid 10% and then dried on anhydrous MgSO₄, filtered and the solvent evaporated. The crude product 3 was loaded on a silica gel open column and eluted with CH₂Cl₂/MeOH (9.5/0.5), from which 4-carbamoylphenyl 2-(4-oxophenyl)-phenyl propanoate (3) was obtained (1.84 g, 42% yield).

Step 2: Synthesis of 4-thiocarbamoylphenyl 2-(4-oxophenyl)-phenyl Propanoate

4-carbamoylphenyl 2-(4-oxophenyl)-phenyl propanoate (3) (1.84 g, 4.93 mmol) and Lawesson reagent (2 g, 4.93 mmol) were dissolved in 100 ml of anhydrous benzene. The reaction was warmed to 60° C. and stirred for 4 h. The solvent was removed under reduced pressure. The obtained compound was purified by a silica gel open column and eluted with CH₂Cl₂/MeOH (9.5/0.5) giving the pure compound 4 (0.45 g, 23% yield).

¹H NMR (DMSO-d6): 8 1.53 (d, 3H), 4.25 (dd, 1H), 7.08 (d, 2H), 7.54-7.73 (m, 9H), 7.90 (d, 2H), 9.51 and 9.88 (s, 2H, NH2). ¹³C NMR (DMSO-d6): 8 19.2, 44.9, 121.6, 129.3, 129.5, 129.8, 130.3, 132.6, 133.5, 137.6, 137.9, 138.1, 141.2, 153.3, 154.5, 156.1, 163.8, 172.9, 199.6.

MS (EI), m/e 390 (M⁺);

m.p: 114-116° C.

### Biological Data

### Example 2: Comparative Evaluation of Ketoprofen and Compound of Formula I, in a Murine Model of Interstitial Cystitis/Visceral Pain Syndrome

### Materials and Methods

### Test Substances and Reagents

Compound of Formula I (ATB-352) was provided by Antibe Therapeutics, Inc., while ketoprofen was purchased from Sigma-Aldrich (Oakville, ON, Canada). Suspensions of compound of Formula I (4.6, 15, 46 or 77 mg kg⁻¹), and molar-equivalent doses of ketoprofen (3, 10, 30 or 50 mg kg1) were freshly prepared in 0.5% CMC; Cromoline Quimica Fina Ltd., Diadema, SP, Brazil). Cyclophosphamide, carbachol, KC1 and CMC were purchased from Sigma Chemical Co. (St. Louis, MO). Isoflurane was obtained from Cristälia Prod Quimicos Farm Ltda. (Itapira/SP, Brazil).

### Animals

Experiments were conducted with male C57BL/6 SPF mice (6-10 weeks of age) obtained from the animal care facilities at the Faculty of Medicine, University of Sao Paulo, Brazil. Mice were randomly separated into different treatment groups (n=5 to 7 per group). Groups of five or fewer mice were kept in Perspex cages under standard controlled conditions (22° C.; 12/12 h light/dark cycle) with free access to food (Nuvilab CR-1, Quimtia S/A, Brazil) and filtered tap water. The study was performed in agreement with the guidelines from the Brazilian Council for Control of Animal Experimentation (CONCEA) guidelines, consistent with the Animal Welfare Act. All the experimental protocols were approved by the local ethics committee (CEUA—ICB; protocol n ° 2055050819). According to the internal laboratory rules, euthanasia was performed if severe distress related to the test agents developed during the experiment.

### Cystitis Induction and Experimental Design

Cystitis was induced by a single i.p. injection of cyclophosphamide CYP (300 mg kg⁻¹; Oliveira et al., 2018), while the control group received an i.p. injection of saline (10 ml kg⁻¹). Twenty-one hours alter injection of CYP, the mice were treated orally with vehicle, ketoprofen or Compound of Formula I. This included a range of doses of ketoprofen (3, 10, 30 or 50 mg kg⁻¹), compound of Formula I (4.6, 15, 46 or 77 mg kg⁻¹) or vehicle (carboxymethylcellulose 0.5%; CMC 0.5%). The drugs were dissolved in 0.5% CMC at a concentration of 10 ml/kg⁻¹. Table 1 lists the treatment groups.

TABLE 1

| Groups and treatments | Dose | N |
|---|---|---|
| Saline + CMC 0.5% | 10 ml kg⁻¹; p.o. | 6 |
| Cystitis + CMC 0.5% | 10 ml kg−1; p.o. | 7 |
| Cystitis + compound of Formula I | 4.6 mg kg⁻¹; p.o. | 6 |
| Cystitis + compound of Formula I | 15 mg kg⁻¹; p.o. | 5 |
| Cystitis + compound of Formula I | 46 mg kg⁻¹; p.o. | 6 |
| Cystitis + compound of Formula I | 77 mg kg⁻¹; p.o. | 6 |
| Cystitis + ketoprofen | 3 mg kg⁻¹; p.o. | 5 |
| Cystitis + ketoprofen | 10 mg kg⁻¹; p.o. | 5 |
| Cystitis + ketoprofen | 30 mg kg⁻¹; p.o. | 5 |
| Cystitis + ketoprofen | 50 mg kg⁻¹; p.o. | 6 |

### Measurement of Mechanical Allodynia via Electronic von Frey Tests

The mice were individually placed in a clear Perspex testing chamber (11 cm×9.5 cm×18 cm) on an elevated wire grid platform (0.5 cm2 grid) for an adequate acclimatization (˜1 h). Mechanical sensitivity assessment was performed with the aid of an electronic von Frey apparatus (Insight Inc., Ribeiräo Preto, SP, Brazil) with groups of 5-7 mice each, as described previously (Costa et al., 2020). Force-generating pressure was applied to the lower abdomen in the vicinity of the bladder (pelvic region between the urethra and the anus) with a pressure transducer connected to a digital force detector (Insight Ltd, Ribeiräo Preto/SP, Brazil). Retraction of the abdomen (mechanical allodynia) from the probe was considered as a positive response to the stimuli and were recorded in grams (g). The stimulus was applied twice with an interstimulus interval of at least 5 to 10 s and the mean of these positive responses (in g) was registered as abdominal withdrawal threshold (mechanical allodynia) and expressed as area-under-the-curve (AUC) over several time-points (21 to 24 hours alter CYP injection). All mechanical sensitivity testing was performed in a blinded manner. Response threshold data were analyzed via analysis of variance (ANOVA) followed by Tukey's post hoc test. All mice were euthanized with an overdose of isoflurane followed by cervical dislocation.

Assessment of gastric lesions in IC/BPS mice treated with ketoprofen versus ATB-352.

The ability of systemic treatment with compound of Formula I (77 mg kg⁻¹, p.o.) and a high equimolar dose of ketoprofen (50 mg kg⁻¹, p.o.) to induce gastric mucosal hemorragic injury, as observed in a previous study (Costa et al., 2020) was studied. Briefly, mice from distinct experimental groups were euthanized by cervical dislocation under isoflurane anesthesia, and the stomach was excised, opened along the smaller curvature, rinsed gently with saline and then photographed with a digital camera (Samsung Galaxy S20, Campinas/SP, Brazil). The hemorrhagic gastric lesion area was measured blindly (mm²) using computerized planimetry software (ImageJ, Maryland, USA).

### Bladder Functional Assay in Vitro

After 24 h, mice with CYP-induced cystitis were euthanized and the bladders were immediately transferred into a petri-dish. The fat tissue was carefully removed, as described by Calmasini et al., 2021. The bladders were divided into two longitudinal strips and mounted in an isolated organ bath system (Panlab Harvard, Massachussetts, USA) containing Krebs Henseleit solution at 37° C. (pH 7.4, O₂/CO₂ 9/5 v/v) under a resting tension of 5 mN. Tissues were allowed to stabilize for 1 h, and during this period the bathing solution was replaced every 15 min. Isometric force was recorded using a PowerLab 8/30 Data Acquisition System (Software Chart, version 6.0, ADInstrument, Milford, MA, USA).

The contractile response of the bladders to a single concentration of KCI (80 mM) or to cumulative increasing carbachol concentrations (10⁻⁹ to 10⁻⁵) were studied in all groups: vehicle (normal animals), CYP control group (untreated animals with interstitial cystits (IC), and the two CYP groups (IC) treated with the test agents (ketoprofen and Compound of Formula I).

### Statistical Analyses

These analyses were performed using the GraphPad Prism® version 6.0 (GraphPad Software, San Diego, USA). The data were analyzed by one-way analysis of variance (ANOVA), followed by Tukey's post hoc test. All the results are presented as mean±standard error of mean (SEM) and the level of significance was set as *P<0.05.

### Results

### Ketoprofen-Based H2S Donor (ATB-352) Exhibited Superior Anti-Hyperalgesic Effects in a Murine Model of Interstitial Cystitis/Visceral (Bladder) Pair Syndrome

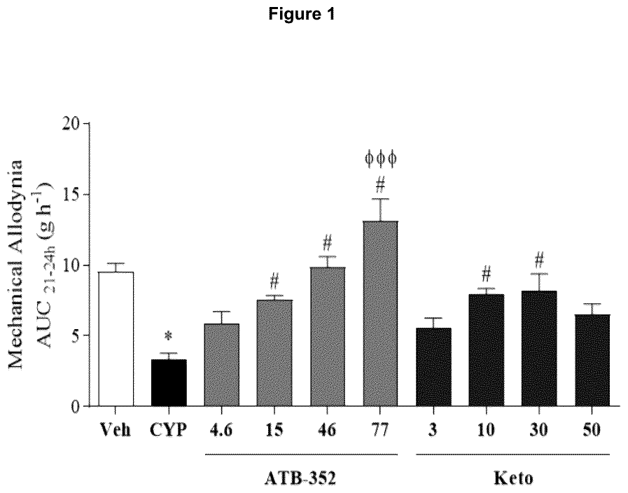

FIG. 1 shows that injection with CYP significantly decreased the nociceptive threshold, expressed as AUC, in the pelvic region of mice orally treated with 0.5% CMC as compared with the control group injected with saline. At the lowest equimolar dose of compound of Formula I or ketoprofen, there was no an anti-hyperalgesic effect on decreased withdrawal threshold in this murine model of IC/BPS. At the higher doses, compound of Formula I reduced cyclophosphamide-induced abdominal mechanical allodynia in a dose-dependent manner. At doses of 10 and 30 mg kg⁻¹, but not 50 mg kg⁻¹, ketoprofen significantly reversed the abdominal mechanical allodynia in CPY-induced IC/BPS. With compound of Formula I (15, 46 and 77 mg kg⁻¹) at intermediate and high equimolar doses, significant dose-dependent differences were detected in the response to (mechanical allodynia) as compared to control IC/BPS mice treated with vehicle 0.5% CMC. Furthermore, at the highest equimolar dose of compound of Formula I (77 mg kg⁻¹) enhanced analgesic effects were observed as compared to ketoprofen (P<0.001; FIG. 1).

### Inhibition of Ketoprofen-Induced Gastric Mucosal Damage by ATB-352

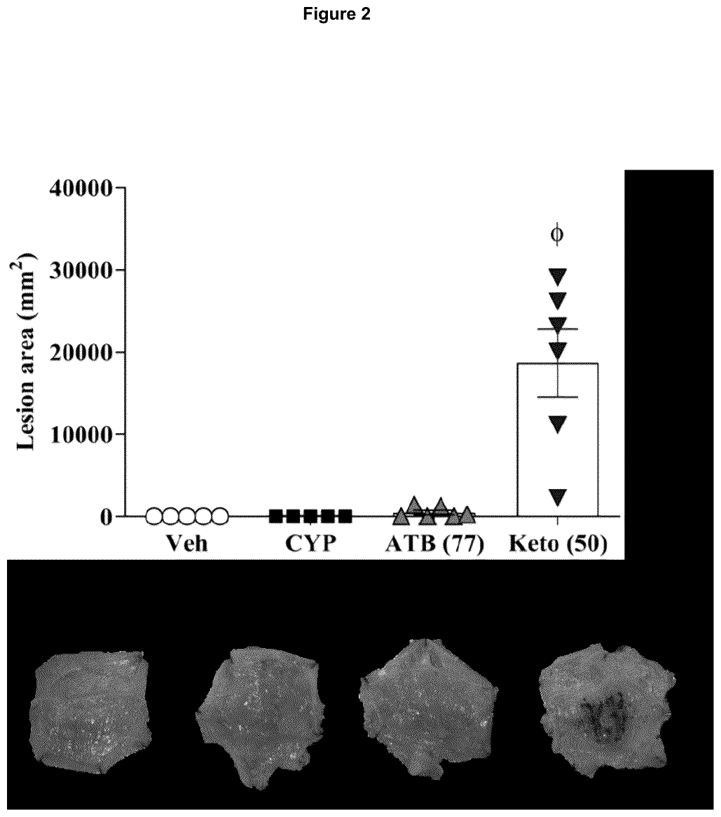

As shown in FIG. 2, post-systemic treatment of mice with a high dose of ketoprofen (50 mg kg⁻¹ p.o.) led to massive gastric lesions in animals over the course of 3 h (FIG. 2 (panel D, fourth picture from left), but treatment of mice with an equimolar dose of compound of Formula I (77 mg kg-1) or vehicle did not (panels B and C, second and third picture from left, respectively).

Panels A to D (first, second, third and fourth pictures from left respectively) of FIG. 2 show pictures of the gastric mucosa of vehicle-treated mice, IC/BPS mice treated with CMC 0.5%, IC/BPS mice treated with the high doses of compound of Formula I and IC/BPS mice treated with the high doses of ketoprofen. Panels A, B and C (first, second and third pictures from left respectively) show a normal gastric mucosa, whilst panel D (fourth picture from left) shows an extensive area of hemorrhagic gastric ulceration.

### Compound of Formula I is Superior to Ketoprofen in Improving of CYP-Induced Reduced Bladder Dysfunction in a Murine Model of IC/BPS

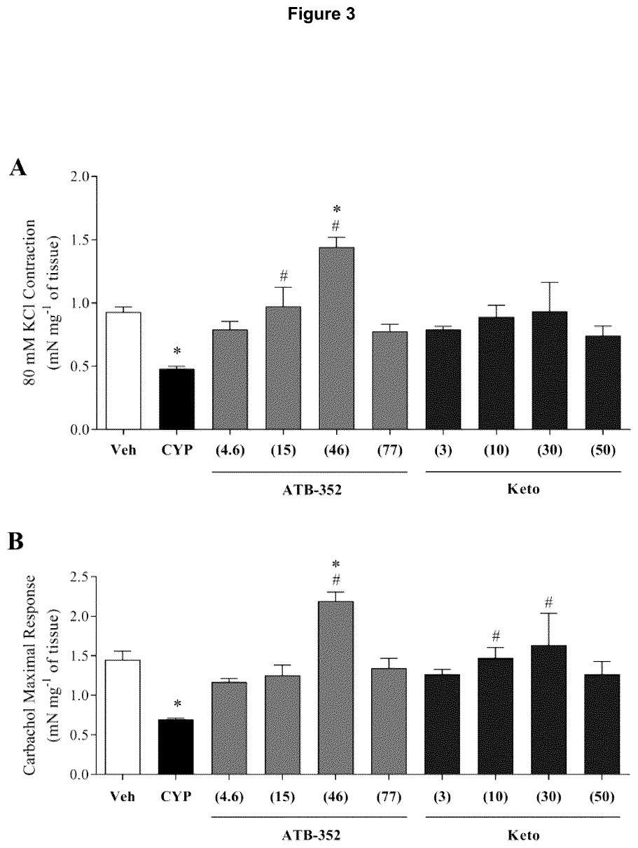

In the control CYP bladder strips, the reduced contractility to KCI (80 mM) was markedly different from the vehicle-treated group (FIG. 3A). However, the reduced contractile response (FIG. 3A) to KCI in CYP bladder strips was significantly and dose-dependently increased in mice treated with compound of Formula I at 15 and 46 mg kg⁻¹, but not with the equimolar doses of ketoprofen, as compared with the control group (p<0.05). At the high doses neither compound of Formula I (77 mg kg⁻¹) nor ketoprofen (50 mg kg⁻¹) significantly improved the reduced bladder contractility in IC/BPS mice in response to KCI.

Similarly, the reduced maximal (Eₘₐₓ) contractile responses elicited by CCh in bladder strips of IC/BPS mice were significantly increased in mice treated with the high doses of compound of Formula I and ketoprofen, but not with the lower doses of each (FIG. 3 B). At the intermediate doses of ketoprofen (10 and 30 mg kg⁻¹), but not compound of Formula I, the reduced maximal (Eₘₐₓ) contractile responses elicited by CCh were significantly reversed (FIG. 3 B).

### Conclusions

The data generated in this study demonstrate that injection of CYP into C57B1/6 mice produces bladder inflammation and related visceral/pelvic pain in addition to reduced functional contractility of isolated bladder phenotype. These functional and behavioral changes in the IC/BPS-like model induced by CYP were greatly diminished by treatment with intermediate and high doses of compound of Formula I, and to a lesser extent by the parent molecule (ketoprofen).

Based on the increased beneficial effects of compound of Formula I compared to ketoprofen on the abdominal hyperalgesic state and reduced bladder contractile response in CYP-induced IC/BPS model, it is concluded that the potency of compound of Formula I is greater than that of ketoprofen when orally administered as a single dose.

Additionally, compound of Formula I was found to reduce the GI toxicity of ketoprofen, while boosting analgesic/anaesthetic effectiveness. Together, these data demonstrate that the H₂S-donating drug, compound of Formula I, may have significant potential for improvement or treatment of urothelial health and symptoms, including visceral pain (bladder inflammation) in patients with IC/BPS.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE SPECIFICATION

A number of publications are cited herein. Full citations for these references are provided below. Each of these references is incorporated herein by reference in its entirety into the present disclosure, to the same extent as if each individual reference was specifically and individually indicated to be incorporated by reference.
Bindu S, Mazumder S, and Bandyopadhyay U. Non-steroidal anti-inflammatory drugs (NSAIDs) and organ damage: a current perspective. Biochem Pharmacol 180: 114147, 2020
Calmasini F B, Alexandre E C, Oliveira M G, Silva F H, Soares A G, Costa S K P, Antunes E. Lipopolysaccharide reduces urethral smooth muscle contractility via cyclooxygenase activation. J Physiol Biochem. 2021 May 21. doi: 10.1007/s13105-021-00819-8.
Clemens J Q, Mullins C, Acherman et al. 2019. Urologic chronic pelvic pain syndrome: insights from the MAPP Research Network. Nature Reviews Urology volume 16, pages187-200. 2.
Costa S K, Muscara M, Allain T, Dallazen J, Santos L G, Buret A G, Vaughan D, Fowler C J, De Nucci G N, Wallace J L. Enhanced analgesic effects and GI safety of a novel hydrogen sulfide-releasing anti-inflammatory drug (ATB-352): A role for endogenous cannabinoids. Antioxid Redox Signal. 2020 Feb. 16. doi: 10.1089/ars.2019.7884.
de Oliveira M G, Calmasini F B, Alexandre E C, De Nucci G, Mónica F Z, Antunes E (2016). Activation of soluble guanylyl cyclase by BAY 58-2667 improves bladder function in cyclophosphamide-induced cystitis in mice. Am J Physiol Renal Physiol. 311: F85-F93.
Glowacka et al., Microbiome Profile and Molecular Pathways Alterations in Gastrointestinal Tract by Hydrogen Sulfide-Releasing Nonsteroidal Anti-Inflammatory Drug (ATB-352): Insight into Possible Safer Polypharmacy ANTIOXIDANTS & REDOX SIGNALING Volume 36, (4-6), 2022
Gugliandolo E, Fusco R, D'Amico R, Militi A, Oteri G, Wallace J L, Di Paola R, and Cuzzocrea S. Antiinflammatory effect of ATB-352, a H₂S-releasing ketoprofen derivative, on lipopolysaccharide-induced periodontitis in rats. Pharmacol Res 132: 220-231, 2018.
Lasheen N N, Elayat W M, Elrefai M F M, Zaki W S, Ahmed E H, El Sheikh R M N, Abo Raya D S A, and Gad F R S. Possible role of garlic oil in ameliorating renal injury after liver ischemia/reperfusion in rats. J Physiol Pharmacol 70: 765-778, 2019.
Malykhina A P. Neural mechanisms of pelvic organ cross-sensitization. Neuroscience. 2007; 149: 660-672. https://doi.org/10.1016/j.neuroscience.2007.07.053 PMID: 17920206.
Okifuji A, Hare B D. The association between chronic pain and obesity. J Pain Res. 2015; 8: 399-408.
Sun H, Yang J, Shi Y, Wang Y, Li C, and Zhu M. Hydrogen sulfide in the nucleus tractus solitarü regulates gastric acid secretion in rats. J Physiol Pharmacol 71: 1-5, 2020.
Russell R I. Non-steroidal anti-inflammatory drugs and gastrointestinal damage—problems and solutions. Postgrad Med J 77: 82-88, 2001.
Wallace J L, Ianaro A, and de Nucci G. Gaseous mediators in gastrointestinal mucosal defense and injury. Dig Dis Sci 62: 2223-2230, 2017.
Winfield R D, Reese S, Bochicchio K, Mazuski J E, Bochicchio G V. Obesity and the risk for surgical site infection in abdominal surgery. Am Surg. 2016; 82: 331-336.

1. A method of treating pain associated with urologic chronic pelvic pain syndrome (UCPPS) in a subject in need thereof comprising administering, to the subject, an effective amount of a compound of Formula I, 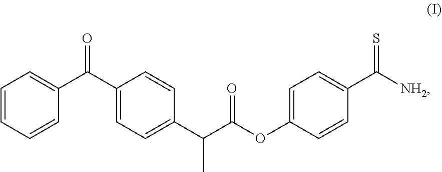 [SMILES: CC(C(=O)Oc1ccc(C(N)=S)cc1)c1ccc(C(=O)c2ccccc2)cc1]
or a pharmaceutically acceptable salt, prodrug and/or solvate thereof.

2. The method of claim 1, wherein the pain associated with UCPPS is selected from one or more of visceral pain, neuropathic pain, neuralgia, allodynia, causalgia, hyperalgesia, hyperesthesia, hyperpathia and neuropathy secondary to surgical procedure.

3. The method of claim 1, wherein the pain associated UCPPS is acute and/or chronic pain associated with UCPPS.

4. The method of claim 3, wherein the pain associated UCPPS is acute pain associated with UCPPS, and the acute pain associated with UCPPS is a transient exacerbation or flare up of pain associated with UCPPS.

5. The method of claim 3, wherein the pain associated UCPPS is acute pain associated with UCPPS, and the acute pain persists for less than about 14 days, less than about 12 days, less than about 10 days, less than about 7 days, less than about 5 days or less than about 3 days from onset of the acute pain.

6. The method of claim 1, wherein the UCPPS is interstitial cystitis/bladder pain syndrome (IC/BPS) or wherein the UCPPS is chronic prostatitis/chronic pelvic pain syndrome (CP/CPPS).

7. A method of treating urologic chronic pelvic pain syndrome (UCPPS) in a subject in need thereof comprising administering, to the subject, an effective amount of a compound of Formula I, 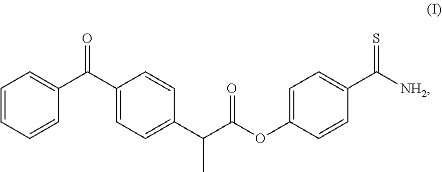 [SMILES: CC(C(=O)Oc1ccc(C(N)=S)cc1)c1ccc(C(=O)c2ccccc2)cc1]
or a pharmaceutically acceptable salt, prodrug and/or solvate thereof.

8. The method of claim 7, wherein the UCPPS is interstitial cystitis/bladder pain syndrome (IC/BPS), or wherein the UCPPS is chronic prostatitis/chronic pelvic pain syndrome (CP/CPPS).

9. A method of treating one or more symptoms associated with urologic chronic pelvic pain syndrome (UCPPS) in a subject in need thereof comprising administering, to the subject, an effective amount of a compound of Formula I, 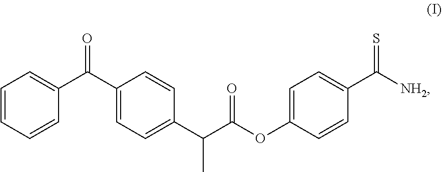 [SMILES: CC(C(=O)Oc1ccc(C(N)=S)cc1)c1ccc(C(=O)c2ccccc2)cc1]
or a pharmaceutically acceptable salt, prodrug and/or solvate thereof.

10. The method of claim 9, wherein the UCPPS is interstitial cystitis/bladder pain syndrome (IC/BPS), or wherein the UCPPS is chronic prostatitis/chronic pelvic pain syndrome (CP/CPPS).

11. The method of claim 9, wherein the one or more symptoms are selected from irritative voiding symptoms, urinary frequency, urinary urgency, nocturia, incontinence and suprapubic or pelvic pain related to and relieved by voiding.

12. A method of treating bladder inflammation in a subject in need thereof comprising administering, to the subject, an effective amount of a compound of Formula I, 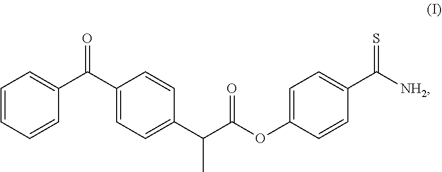 [SMILES: CC(C(=O)Oc1ccc(C(N)=S)cc1)c1ccc(C(=O)c2ccccc2)cc1]
or a pharmaceutically acceptable salt, prodrug and/or solvate thereof.

13. The method of claim 12, wherein the bladder inflammation is associated with urologic chronic pelvic pain syndrome (UCPPS).

14. A method of increasing urinary bladder contraction and relaxation in a subject in need thereof comprising administering, to the subject, an effective amount of a compound of Formula I, 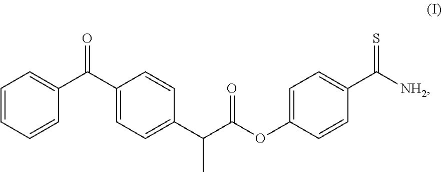 [SMILES: CC(C(=O)Oc1ccc(C(N)=S)cc1)c1ccc(C(=O)c2ccccc2)cc1]
or a pharmaceutically acceptable salt, prodrug and/or solvate thereof.

15. The method of claim 14, wherein the subject has interstitial cystitis/bladder pain syndrome (IC/BPS) or wherein the subject has chronic prostatitis/chronic pelvic pain syndrome (CP/CPPS).

16. A method of treating acute pain in a subject in need thereof comprising administering, to the subject, an effective amount of a compound of Formula I, 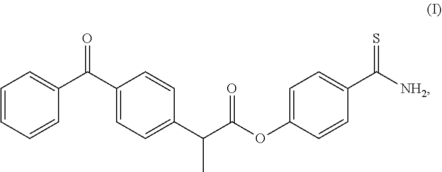 [SMILES: CC(C(=O)Oc1ccc(C(N)=S)cc1)c1ccc(C(=O)c2ccccc2)cc1]
or a pharmaceutically acceptable salt, prodrug and/or solvate thereof.

17. The method of claim 16, wherein the acute pain is a transient exacerbation or flare up.

18. The method of claim 16, wherein the acute pain is post-operative pain resulting from abdominal and/or pelvic surgery.

19. The method of claim 16, wherein the compound of Formula I or a pharmaceutically acceptable salt, prodrug and/or solvate thereof is administered in oral dosages of from about 50 mg per day to about 500 mg per day for an adult.

20. The method of claim 16, wherein the compound of Formula I or a pharmaceutically acceptable salt, prodrug and/or solvate thereof is administered in oral dosages ranging from about 200 mg per day to about 400 mg per day, about 250 mg per day to about 350 mg per day or about 250 mg per day to about 325 mg per day and the compound of Formula I or a pharmaceutically acceptable salt, prodrug and/or solvate thereof is administered for 1 day to 10 days, 1 day to 8 days, 1 day to 7 days, 1 day to 5 days, 2 days to 7 days, 3 days to 7 days, 4 days to 7 days, 5 days to 7 days, 6 days to 7 days, 2 days to 5 days, 3 days to 5 days, 4 days to 5 days, 2 days, 3 days, 4 days, 5 days, 6 days or 7 days.
